(19) United States
(12) Patent Application Publication
(10) Pub. No.: US 2025/0287946 A1
(43) Pub. Date: Sep. 18, 2025

(54) MODIFIED RELEASE OF ASCAROSIDES

(71) Applicant: Ascribe Bioscience Inc., Ithaca, NY (US)

(72) Inventor: Jay Farmer, Ithaca, NY (US)

(21) Appl. No.: 18/860,035

(22) PCT Filed: Apr. 28, 2023
(86) PCT No.: PCT/US23/20472

§ 371 Date: Oct. 25, 2024

Related U.S. Application Data
(60) Provisional application No. 63/336,738, filed on Apr. 29, 2022.

(51) Int. Cl. A01N 43/16; A01P 21/00; C08B 37/00

(52) U.S. Cl. CPC A01N 43/16; A01P 21/00; C08B 37/0006

(57) ABSTRACT

This application relates to modified release compounds and compositions comprising one or more ascarosides, which can be applied to plants to provide increased disease resistance. Certain compounds are structurally modified ascarosides, e.g., in the form of ascaroside-containing polymers. Certain compounds are physically modified ascarosides in the form of one or more ascarosides compounded with organic and/or inorganic components.

### CROSS-REFERENCE TO RELATED APPLICATIONS

This application claims priority to U.S. Provisional Patent Application Ser. No. 63/336,738 entitled “Modified Release of Ascarosides” filed on Apr. 29, 2022, which is incorporated herein by reference.

### FIELD OF THE INVENTION

This application generally relates to agrichemical compounds, compositions and methods of treating plants to promote resistance to pathogens.

### BACKGROUND OF THE INVENTION

Ascaroside natural products are secondary metabolites produced by nematodes. A large number of structurally diverse ascarosides have been identified in nature and the molecules are believed to function as an evolutionarily conserved chemical language used by nematodes to control many aspects of their development. Ascarosides are also perceived by other organisms and have been demonstrated to have a range of effects on numerous organisms including bacteria, fungi, plants, and mammals including humans. Ascarosides hold potential as human medicines, agrichemicals and products for other diverse and valuable applications.

Ascaroside treatments have been demonstrated to increase plant resistance to certain pathogens by inducing or priming one or more plant defense responses (which can inhibit pathogenic growth and/or infestation) when applied to the plant. By activating and/or priming plants' innate defenses, ascarosides can thereby prevent the proliferation of pathogens and/or protect crops from the damaging effects of pathogens.

Application of agrichemicals to plants and surrounding soil generally suffers from various disadvantages, such as runoff/drift. It would be useful to provide compositions and methods which can provide for modified release of these compounds to plants during growth.

### SUMMARY OF THE INVENTION

The disclosure provides compositions and methods for modified release of ascarosides. In some embodiments, such modified release is effected by making structural changes to the ascaroside molecule. In other embodiments, such modified release is provided by composition-based approaches, which may or may not result in structural changes to the ascaroside(s) contained within the formulation. The disclosure further provides methods for extending the time during which ascaroside-based treatments can affect the modulation of disease resistance in plants.

Ascarosides are biodegradable and have been shown to be rapidly broken down in soil and to be metabolized by other organisms, including plants. This presents challenges for some commercial uses of ascarosides. For example, in an agricultural setting, it may be desirable to apply ascarosides as seed treatments or to provide them via in-furrow applications when crops are planted, in pharmaceutical applications, it may be desirable to develop time release or implantable devices that release ascarosides over time. In the case of uses on plants, the inventors have observed that such applications are not always reliable and hypothesize that this is because the ascaroside molecules break down before they can be taken up by crop plants. For example, if a seed that is coated with a seed treatment containing an unmodified ascaroside takes a week or more to germinate, there is a risk that much of the ascaroside in the coating will biodegrade while in contact with the soil before the plant emerges from the seed. This may result in the intended ascaroside-based response in the germinating plant not being achieved or being attenuated. To solve this problem, the present invention provides ascaroside compositions that are less prone to rapid biodegradation and/or that are designed to release ascarosides over an extended period of time. This delayed or extended release increases the opportunity for crop plants to absorb or respond to the ascaroside and improves the reliability and/or convenience of applying such products.

The present disclosure includes, without limitation, the following embodiments.

Embodiment 1: A structurally modified ascaroside, selected from the group consisting of: a) an ascaroside-based oligomer or polymer, comprising two or more ascaroside moieties, optionally comprising linker moieties between adjacent ascaroside moieties; b) an oligomer- or polymer-functionalized ascaroside, comprising an ascaroside moiety with one, two, or three oligomers or polymers attached thereto; and c) an ascaroside-pendant polymer, comprising a polymer with one or more pendant ascaroside moieties attached thereto, optionally comprising linker moieties between the polymer and the pendant ascaroside moieties.

Embodiment 2: The structurally modified ascaroside of Embodiment 1, wherein the modified ascaroside is an ascaroside-based oligomer or polymer, and wherein the oligomer or polymer comprises two or more ascaroside moieties with no linker moieties between adjacent ascaroside moieties.

Embodiment 3: The structurally modified ascaroside of Embodiment 1 or 2, wherein the modified ascaroside is an ascaroside-based oligomer or polymer, and wherein adjacent ascaroside moieties are attached to one another via an ester linkage formed from a hydroxyl group on a first ascaroside molecule and a carboxylic acid on a second ascaroside molecule.

Embodiment 4: The structurally modified ascaroside of Embodiment 1, wherein the modified ascaroside is an ascaroside-based oligomer or polymer, and wherein the oligomer or polymer comprises two or more ascaroside moieties with linker moieties between adjacent ascaroside moieties.

Embodiment 5: The structurally modified ascaroside of Embodiment 1, wherein the linker moieties are selected from the group consisting of ether, ester, amide, glycosidic, urethane, carbonate, and siloxano moieties.

Embodiment 6: The structurally modified ascaroside of any of Embodiments 1 to 5, wherein the modified ascaroside is an ascaroside-based oligomer or polymer wherein the oligomer or polymer comprises two to one thousand ascaroside moieties.

Embodiment 7: The structurally modified ascaroside of Embodiment 6, wherein the oligomer or polymer comprises two to five hundred ascaroside moieties.

Embodiment 8: The structurally modified ascaroside of Embodiment 6, wherein the oligomer or polymer comprises two to one hundred ascaroside moieties.

Embodiment 9: The structurally modified ascaroside of Embodiment 6, wherein the oligomer or polymer comprises two to ten ascaroside moieties.

Embodiment 10: The structurally modified ascaroside of Embodiment 6, wherein the oligomer or polymer comprises at least ten ascaroside moieties.

Embodiment 11: The structurally modified ascaroside of Embodiment 6, wherein the oligomer or polymer comprises at least twenty ascaroside moieties.

Embodiment 12: The structurally modified ascaroside of Embodiment 6, wherein the oligomer or polymer comprises at least fifty ascaroside moieties.

Embodiment 13: The structurally modified ascaroside of Embodiment 1, wherein the modified ascaroside is an oligomer- or polymer-functionalized ascaroside, comprising one oligomer or polymer attached to the ascaroside.

Embodiment 14: The structurally modified ascaroside of Embodiment 1, wherein the modified ascaroside is an oligomer- or polymer-functionalized ascaroside, comprising two oligomers or polymers attached to the ascaroside.

Embodiment 15: The structurally modified ascaroside of Embodiment 1, wherein the modified ascaroside is an oligomer- or polymer-functionalized ascaroside, comprising three oligomers or polymers attached to the ascaroside.

Embodiment 16: The structurally modified ascaroside of Embodiment 1, wherein the modified ascaroside is an ascaroside-pendant polymer, comprising about five to about five hundred pendant ascaroside moieties attached thereto.

Embodiment 17: The structurally modified ascaroside of Embodiment 1 or 16, wherein the modified ascaroside is an ascaroside-pendant polymer, and wherein the one or more pendant ascaroside moieties are directly bonded to the polymer.

Embodiment 18: The structurally modified ascaroside of Embodiment 1 or 16, wherein the modified ascaroside is an ascaroside-pendant polymer, comprising linker moieties between the polymer and the one or more pendant ascaroside moieties

Embodiment 19: The structurally modified ascaroside of Embodiment 18, wherein the linker moieties are selected from the group consisting of ether, ester, amide, glycosidic, urethane, carbonate, and siloxane moieties

Embodiment 20: The structurally modified ascaroside of any of Embodiments 13-19, wherein the oligomer or polymer comprises a biodegradable polymer.

Embodiment 21: The structurally modified ascaroside of any of Embodiments 13-20, wherein the oligomer or polymer is selected from the group consisting of chitosan, chitin, starches, lignin, agar, pectin, cellulose, other polysaccharides (e.g., bark, sawdust, other cellulosic waste), poly(ethylene glycol), poly(caprolactone), poly(lactide), poly(glycolic acid), polybutylene, poly(vinyl alcohol), poly(vinyl chloride), poly(citric acid), poly(acrylate), poly(aspartic acid), poly(ethylene), polyethylene vinyl acetate (PEVA), polystyrene, divinyl benzene, and derivatives and copolymers thereof.

Embodiment 22: The structurally modified ascaroside of any of Embodiments 1-21, wherein the ascaroside moieties have the structure (I)

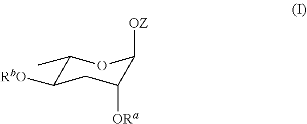

where:
Z is an optionally substituted C₃₋₄₀ aliphatic group, and each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g.—a moiety —C(O)OR^{c}), a C₂₋₂₀ carbamate (e.g.—a moiety —C(O)N(R^{c})₂), a C₂₋₂₀ thioester (e.g. a moiety) —C(S)R^{c}), a C₂₋₂₀ thiocarbonate (e.g. a moiety —C(S)OR^{c}), a C₂₋₂₀ dithiocarbonate (e.g. a moiety —C(S)SR^{c}), a C₁₋₂₀ thiocarbamate (e.g. a moiety —C(S)N R^{c})₂), a sugar moiety, a peptide, a polymer chain, or a linkage via a bond or a carbon-containing linker moiety to another ascaroside molecule, where R^{c}is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule, and where R^{a} and R^{b} may be taken together to form an optionally substituted ring, optionally containing one or more heteroatoms, and optionally containing one or more sites of unsaturation.

Embodiment 23: The structurally modified ascaroside of Embodiment 22, wherein Z is selected from the group consisting of:
i. —CH(CH₃)—R¹, where R¹ is an optionally substituted C₁₋₄₀ aliphatic group
ii. —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide;
iii. —CH(CH₃)—(CH₂)ₙ—CH═CH—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide;
iv. —CH(CH₃)—(CH₂)ₙ—CH(OH)—CH—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide;
v. —CH(CH₃)—(CH₂)ₙ—C(O)—CH—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide;
vi. —(CH₂)ₙ—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide;
vii. —(CH₂)ₙCH═CH—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide;
viii. —(CH₂)ₙ—CH(OH)—CH—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide;
ix. —(CH₂)ₙ—C(O)—CH—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide;
x. —CH(CH₃)—(CH₂)ₙ—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xi. —CH(CH₃)—(CH₂)ₙ—CH═CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xii. —CH(CH₃)—(CH₂)ₙ—CH(OH)—CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xiii. —CH(CH₃)—(CH₂)ₙ—C(O)—CH—CON(R³)₂, where n is an integer 1 to 40, and each R³ is independently—H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xiv. —(CH₂)ₙ—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xv. —(CH₂)ₙ—CH═CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;

xvi.-(CH₂)ₙ—CH(OH)—CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule; or
xvii. —(CH₂)ₙ—C(O)—CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule.

Embodiment 24: The structurally modified ascaroside of Embodiment 23, wherein R^{a} and R^{b} are each —H

Embodiment 25: The structurally modified ascaroside of any of Embodiments 22-24, wherein Z is —CH(CH₃)—(CH₂)ₙ—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide

Embodiment 26: The structurally modified ascaroside of any of Embodiments 1-25, wherein at least one of the ascaroside moieties is ascr#18

Embodiment 27: A physically modified ascaroside, comprising one or more ascarosides compounded with an organic or inorganic material.

Embodiment 28: The physically modified ascaroside of Embodiment 27, wherein the ascaroside is compounded with an organic material selected from coir, peat, and/or pine bark.

Embodiment 29: The physically modified ascaroside of Embodiment 27, wherein the ascaroside is compounded with a polymeric resin.

Embodiment 30: The physically modified ascaroside of Embodiment 29, wherein the polymeric resin is a biodegradable polymer.

Embodiment 31: The physically modified ascaroside Embodiment 29 or 30, wherein the polymeric resin is a melt-processable polymer.

Embodiment 32: The physically modified ascaroside of any of Embodiments 29-31, wherein the polymeric resin is selected from the group consisting of polypropylene, polyethylene, poly(vinyl chloride), polystyrene, polyethylene terephthalate, polyethylene-vinyl acetate, polyurethane, acrylonitrile butadiene styrene (ABS), nylon, plant starch, polylactic acid (PLA), polyhydroxyalkanoate (PHA), and derivatives and co-polymers of any two or more thereof.

Embodiment 33: The physically modified ascaroside of Embodiment 27, wherein the ascaroside is compounded with an inorganic material selected from the group consisting of perlite, vermiculite, sand, clay, talc, wood flour, peat, silica gel, mica, activated charcoal, and combinations thereof.

Embodiment 34: The physically modified ascaroside of Embodiment 27, wherein the ascaroside is compounded with an inorganic material selected from a fertilizer (e.g., ammonium phosphate, ammonium sulfate, urea, muriate of potash, superphosphate), herbicides (e.g., sodium chlorate or sodium sulfate), insoluble silica-type material (e.g., sand or broken walnut hulls), argillaceous materials (e.g., clay of the montmorillonite, kaolinite, or other type), diatomaceous earths, granulated corn cobs, soapstone, quartz, lead, iron, and agricultural minerals (e.g., sulfur, gypsum, lime, calcium carbonate, and the like), and combinations thereof.

Embodiment 35: The physically modified ascaroside of any one of Embodiments 27-34, comprising one or more bonds between one or more of the ascarosides and the organic or inorganic material.

Embodiment 36: The physically modified ascaroside of any one of Embodiments 27-36, further comprising one or more additional components selected from the group consisting of surfactants, including emulsifiers, dispersants, foam-formers, colorants, processing aids, lubricants, fillers, reinforcements, flame retardants, light stabilizers, ultraviolet radiation absorbers, weather stabilizers, plasticizers, release agents, perfumes, heat-retaining additives (e.g., silica), cross-linking agents, antioxidants, anti-foaming agents, buffers, pH modifiers, compatibility agents, drift control additives, extenders/stickers, tackifiers, plant penetrants, safeners, spreaders, and wetting agents.

Embodiment 37: The physically modified ascaroside of any of Embodiments 27-36, wherein at least one of the ascarosides has the structure (I)

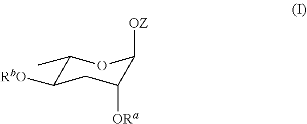

where:
Z is an optionally substituted C₃₋₄₀ aliphatic group, and
each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g. —a moiety —C(O)OR^{c}), a C₂₋₂₀ carbamate (e.g.—a moiety —C(O)N(R^{c})₂), a C₂₋₂₀ thioester (e.g. a moiety —C(S)R^{c}), a C₂₋₂₀ thiocarbonate (e.g. a moiety —C(S)OR^{c}), a C₂₋₂₀ dithiocarbonate (e.g. a moiety —C(S)SR^{c}), a C₁₋₂₀ thiocarbamate (e.g. a moiety —C(S)N(R^{c})₂), a sugar moiety, a peptide, a polymer chain, or a linkage via a bond or a carbon-containing linker moiety to another ascaroside molecule. Where R^{c} is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule, and where R^{a} and R^{b} may be taken together to form an optionally substituted ring, optionally containing one or more heteroatoms, and optionally containing one or more sites of unsaturation.

Embodiment 38: The physically modified ascaroside of Embodiment 37, wherein Z is selected from the group consisting of:
i. —CH(CH₃)—R¹, where R¹ is an optionally substituted C₁₋₄₀ aliphatic group
ii. —CH(CH₃)—(CH₂)ₙ—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide;
iii. —CH(CH₃)—(CH₂)ₙ—CH═CH—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide;
iv. —CH(CH₃)—(CH₂)ₙ—CH(OH)—CH—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide;
v. —CH(CH₃)—(CH₂)ₙ—C(O)—CH—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide;
vi. —(CH₂)ₙ—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide;
vii. —(CH₂)ₙ—CH═CH—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide;
viii. —(CH₂)ₙ—CH(OH)—CH—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide;
ix. —(CH₂)ₙ—C(O)—CH—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide;
x. —CH(CH₃)—(CH₂)ₙ—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xi. —CH(CH₃)—(CH₂)ₙ—CH═CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xii. —CH(CH₃)—(CH₂)ₙ—CH(OH)—CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xiii. —CH(CH₃)—(CH₂)ₙ—C(O)—CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xiv. —(CH₂)ₙ—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xv. —(CH₂)ₙ—CH═CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xvi. —(CH₂)ₙ—CH(OH)—CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule; or
xvii. —(CH₂)ₙ—C(O)—CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule.

Embodiment 39: The physically modified ascaroside of Embodiment 37 or 38, wherein R^{a} and R^{b} are each —H

Embodiment 40: The physically modified ascaroside of any one of Embodiments 37-39, wherein Z is —CH(CH₃)—(CH₂)ₙ—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide.

Embodiment 41: The physically modified ascaroside of any one of Embodiments 27-40, wherein at least one of the ascaroside moieties is ascr#18.

Embodiment 42: A composition comprising the structurally modified ascaroside of any one of Embodiments 1-26 and/or the physically modified ascaroside of any one of Embodiments 27-41 and one or more agronomically acceptable excipients or additives.

Embodiment 43: The composition of Embodiment 42, in the form of a liquid.

Embodiment 44: The composition of Embodiment 43, wherein the liquid is a sprayable formulation.

Embodiment 45: The composition of Embodiment 42, in the form of a solid.

Embodiment 46: The composition of Embodiment 45, wherein the solid comprises granules.

Embodiment 47: The composition of Embodiment 45, wherein the solid comprises plastic mulch.

Embodiment 48: The composition of Embodiment 45, wherein the solid composition is a film.

Embodiment 49: A method, comprising: contacting a plant or portion thereof or soil surrounding the plant or portion thereof with the structurally modified ascaroside of any of Embodiments 1-26, the physically modified ascaroside of any of Embodiments 27-41, or the composition of any of Embodiments 42-48, wherein the ascaroside or composition is effective to increase plant resistance to one or more pathogens and/or to induce an immune response in the plant or portion thereof.

Embodiment 50: The method of Embodiment 49, wherein the portion thereof is selected from the group consisting of root, stem, leaf, seed, and flower.

Embodiment 51: The method of Embodiment 49 or 50, wherein the plant is a vegetable or fruit plant.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise. Other aspects and advantages of the present disclosure will become apparent from the following.

### DEFINITIONS

In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. As used in this application, the term “comprise” and variations of the term, such as “comprising” and “comprises,” are not intended to exclude other additives, components, integers or steps.

About, Approximately: As used herein, the terms “about” and “approximately” are used as equivalents. Unless otherwise stated, the terms “about” and “approximately” may be understood to permit standard variation as would be understood by those of ordinary skill in the art. Where ranges are provided herein, the endpoints are included. Any numerals used in this application with or without about/approximately are meant to cover any normal fluctuations appreciated by one of ordinary skill in the relevant art. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

Certain compounds provided herein can comprise one or more asymmetric centers, and thus can exist in various stereoisomeric forms, e.g., enantiomers and/or diastereomers. Thus, inventive compounds and compositions thereof may be in the form of an individual enantiomer, diastereomer or geometric isomer, or may be in the form of a mixture of stereoisomers. In certain embodiments, compounds described herein are enantiopure compounds. In certain other embodiments, mixtures of enantiomers or diastereomers are provided.

Furthermore, certain compounds as described herein may have one or more double bonds that can exist as either a Z or E isomer, unless otherwise indicated. The compounds can be provided as individual isomers substantially free of other isomers and alternatively, as mixtures of various isomers, e.g., racemic mixtures of enantiomers.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the disclosure. For instance, a compound may, in some embodiments, be provided substantially free of one or more corresponding stereoisomers, and may also be referred to as “stereochemically enriched.”

Where a particular enantiomer is preferred, it may, in some embodiments be provided substantially free of the opposite enantiomer, and may also be referred to as “optically enriched.” “Optically enriched,” as used herein, means that the compound is made up of a significantly greater proportion of one enantiomer. In certain embodiments the compound is made up of at least about 90% by weight of an enantiomer. In some embodiments the compound is made up of at least about 95%, 97%, 98%, 99%, 99.5%, 99.7%, 99.8%, or 99.9% by weight of an enantiomer. In some embodiments the enantiomeric excess of provided compounds is at least about 90%, 95%, 97%, 98%, 99%, 99.5%, 99.7%, 99.8%, or 99.9%. In some embodiments, enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, IN 1972).

The terms “halo” and “halogen” as used herein refer to an atom selected from fluorine (fluoro, —F), chlorine (chloro, —Cl), bromine (bromo, —Br), and iodine (iodo, —I).

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-30 carbon atoms. In certain embodiments, aliphatic groups contain 1-12 carbon atoms. In certain embodiments, aliphatic groups contain 1-8 carbon atoms. In certain embodiments, aliphatic groups contain 1-6 carbon atoms. In some embodiments, aliphatic groups contain 1-5 carbon atoms, in some embodiments, aliphatic groups contain 1-4 carbon atoms, in yet other embodiments aliphatic groups contain 1-3 carbon atoms, and in yet other embodiments aliphatic groups contain 1-2 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “heteroaliphatic” or “heteroaliphatic group”, as used herein, denotes an aliphatic group where one or more carbon or hydrogen atoms are replaced by a heteroatom (e.g. oxygen, nitrogen, sulfur, phosphorous, boron, etc.).

The term “unsaturated”, as used herein, means that a moiety has one or more double or triple bonds.

The term “alkyl,” as used herein, refers to saturated, straight- or branched-chain hydrocarbon radicals derived from an aliphatic moiety containing between one and six carbon atoms by removal of a single hydrogen atom. Unless otherwise specified, alkyl groups contain 1-12 carbon atoms. In certain embodiments, alkyl groups contain 1-8 carbon atoms. In certain embodiments, alkyl groups contain 1-6 carbon atoms. In some embodiments, alkyl groups contain 1-5 carbon atoms, in some embodiments, alkyl groups contain 1-4 carbon atoms, in yet other embodiments alkyl groups contain 1-3 carbon atoms, and in yet other embodiments alkyl groups contain 1-2 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

The term “alkenyl,” as used herein, denotes a monovalent group derived from a straight- or branched-chain aliphatic moiety having at least one carbon-carbon double bond by the removal of a single hydrogen atom. Unless otherwise specified, alkenyl groups contain 2-12 carbon atoms. In certain embodiments, alkenyl groups contain 2-8 carbon atoms. In certain embodiments, alkenyl groups contain 2-6 carbon atoms. In some embodiments, alkenyl groups contain 2-5 carbon atoms, in some embodiments, alkenyl groups contain 2-4 carbon atoms, in yet other embodiments alkenyl groups contain 2-3 carbon atoms, and in yet other embodiments alkenyl groups contain 2 carbon atoms. Alkenyl groups include, for example, ethenyl, propenyl, butenyl, 1-methyl-2-buten-1-yl, and the like.

The term “aryl” used alone or as part of a larger moiety as in “aralkyl”, “aralkoxy”, or “aryloxyalkyl”, refers to monocyclic and polycyclic ring systems having a total of five to 20 ring members, wherein at least one ring in the system is aromatic and wherein each ring in the system contains three to twelve ring members. The term “aryl” may be used interchangeably with the term “aryl ring”. In certain embodiments, “aryl” refers to an aromatic ring system which includes, but is not limited to, phenyl, biphenyl, naphthyl, anthracyl and the like, which may bear one or more substituents. Also included within the scope of the term “aryl”, as it is used herein, is a group in which an aromatic ring is fused to one or more additional rings, such as benzofuranyl, indanyl, phthalimidyl, naphthimidyl, phenantriidinyl, or tetrahydronaphthyl, and the like.

As described herein, compounds as provided herein may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{○}; —(CH₂)₀₋₄OR^{○}; —O—(CH₂)₀₋₄C(O)OR^{○}; —(CH₂)₀₋₄CH(OR^{○})₂; (CH₂)₀₋₄SR^{○}; —(CH₂)₀₋₄Ph, which may be substituted with R^{○}; —(CH₂)₀₋₄₀(CH₂)₀₋₁Ph which may be substituted with R^{○}; —CH═CHPh, which may be substituted with R^{○}; —NO₂; —CN; —N₃; —(CH₂)₀₋₄N (R^{○})₂; —(CH₂)₀₋₄N(R^{○}C(O)R^{○}; —N R^{○}C(S)R^{○}; —(CH₂)₀₋₄N(R^{○})C(O)NR^{○}₂; —N (R^{○})C(S)NR^{○}₂; —(CH₂)₀₋₄N(R^{○})C(O)OR^{○}; —N(R^{○})N(R^{○})C(O)R^{○}; —N(R^{○})N(R^{○})C(O)NR^{○}₂; —N(R^{○})N(R^{○})C(O)OR^{○}; —(CH₂)₀₋₄C(O)R^{○}; —C(S)R^{○}; —(CH₂)₀₋₄C(O)OR^{○}; —(CH₂)₀₋₄C(O)N(R^{○})₂; —(CH₂)₀₋₄C(O)SR^{○}; —(CH₂)₀₋₄C(O)OSiR^{○}₃; —(CH₂)₀₋₄OC(O)R^{○}; —OC(O)(CH₂)₀₋₄SR—, SC(S)SR^{○}; —(CH₂)₀₋₄SC(O)R^{○}; —(CH₂)₀₋₄C(O)NR^{○}₂; —C(S)NR^{○}₂; —C(S)SR^{○}; —SC(S)SR^{○}, —(CH₂)₀₋₄OC(O)NR^{○}₂; —C(O)N(OR^{○})R^{○}; —C(O)C(O)R^{○}; —C(O)CH₂C(O)R^{○}; —C(NOR^{○})R^{○}; —(CH₂)₀₋₄SSR^{○}; —(CH₂)₀₋₄S(O)₂R^{○}; —(CH₂)₀₋₄S(O)₂OR^{○}; —(CH₂)₀₋₄OS(O)₂R^{○}; —S(O)₂NR^{○}₂; —(CH₂)₀₋₄S(O)R^{○}; —N(R^{○}S(O)₂NR^{○}₂; —N(R^{○}S(O)₂R^{○}; —N(OR═)R^{○}; —C(NH)NR^{○}₂; —P(O)₂R^{○}; —P(O)R^{○}₂; —OP(O)R^{○}₂; —OP(O)(OR^{○})₂; SiR^{○}₃; —(C₁₋₄ straight or branched alkylene) O—N(R^{○})₂; or -(C₁₋₄ straight or branched alkylene) C(O)O—N(R^{○})₂, wherein each R^{○} may be substituted as defined below and is independently hydrogen, C₁₋₈ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{○}, taken together with their intervening atom(s), form a 3-12-membered saturated, partially unsaturated, or aryl mono- or polycyclic ring having 0-4 heteroatoms independently selected from nitrogen, oxygen, or sulfur, which may be substituted as defined below.

Suitable monovalent substituents on R^{○} (or the ring formed by taking two independent occurrences of R^{○} together with their intervening atoms), are independently halogen, —(CH₂)₀₋₂R·, —(haloR·), —(CH₂)₀₋₂OH, —(CH₂)₀₋₂OR·, —(CH₂)₀₋₂CH(OR·)₂; —O(haloR·) , —CN, —N₃, —(CH₂)₀₋₂C(O)R·, —(CH₂)₀₋₂C(O)OH, —(CH₂)₀₋₂C(O)OR·, —(CH₂)₀₋₄C(O)N(R^{○})₂; —(CH₂)₀₋₂SR·, —(CH₂)₀₋₂SH, —(CH₂)₀₋₂NH₂, —(CH₂)₀₋₂NHR·, —(CH₂)₀₋₂NR·₂, —NO₂, —SiR·₃, —OSiR·₃, —C(O)SR·, —(C₁₋₄ straight or branched alkylene) C(O)OR·, or —SSR· wherein each R· is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{○} include ═O and ═S.

Suitable divalent substituents on a saturated carbon atom of an “optionally substituted” group include the following: ═O, ═S, ═NNR*₂, =NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R·, —(haloR·), —OH, —OR·, —O(haloR·), —CN, —C(O)OH, —C(O)OR·, —NH₂, —NHR·, —NR·₂, or —NO₂, wherein each R· is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{\}₂, —C(S)NR^{\}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{\}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R·, —(haloR·), —OH, —OR·, —O(haloR·), —CN, —C(O)OH, —C(O)OR^{†}, —NH₂, —NHR^{†}, —NR^{†}₂, or —NO₂, wherein each R^{†} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “substantially” refers to the qualitative condition of exhibiting total or near-total extent or degree of a characteristic or property of interest.

The convention of naming ascarosides by a several-letter prefix followed by a pound sign (#) and a number is sometimes used (for example ascr#18). This convention is used in the scientific literature and the skilled artisan will understand that cach such name is associated with a specific chemical structure of known composition and will readily apprehend the structure of the molecule referred to using this naming convention. Unless otherwise indicated, all compound identifiers of this format used herein conform to the definitions described in the C. elegans Small Molecule Identifier Database (SMID-DB) maintained at http://www.smid-db.org.

The term “pathogen” refers to any bacterium, fungus, oomecyte, virus, nematode (e.g., cyst or root knot nematode) or insect with pathogenic effects on a plant.

### BRIEF DESCRIPTION OF THE DRAWINGS

In the drawings, the features are not necessarily represented to scale, emphasis instead generally being placed upon illustrating the principles of the disclosed compositions and methods and are not intended as limiting. For purposes of clarity, not every component may be labeled in the drawing. In the following description, various embodiments are described with reference to the following drawings, in which:

FIGS. 1A, 1B, 1C, and 1D are non-limiting representations of examples of ascaroside oligomers according to certain embodiments of the present disclosure;

FIG. 2 is a non-limiting representation of an oligomeric or polymeric modified ascaroside comprising ester linkages between adjacent ascaroside moieties;

FIGS. 3A and 3B are non-limiting representations of examples of dimeric ascarosides with no linker there between;

FIGS. 4A, 4B, and 4C are non-limiting representations of ascaroside moieties comprising one, two, and three polymeric arms, respectively; and

FIG. 5 is a non-limiting representation of a polymer comprising pendant ascaroside moieties.

### DETAILED DESCRIPTION OF THE INVENTION

Compounds, compositions, and methods for time release of ascarosides are provided. The disclosure is directed to delivering ascarosides or derivatives thereof via modified release profiles. The modified release profiles can be, e.g., extended, delayed, and/or controlled release, useful to maintain an effective concentration of the ascarosides or derivatives thereof over an extended period of time. Some such modified release profiles can be provided via modifications to the structure of the ascaroside (or a derivative thereof), such that the desired ascaroside molecule or its derivative is released from the modified composition over time and/or upon certain environmental exposures/triggers. Some such modified release profiles can be provided via composition-based approaches, such that the desired ascaroside molecule or derivative thereof is released based on degradation of a composition within which it is contained.

Ascarosides are secondary metabolites produced by nematodes. A large number of structurally diverse ascarosides have been identified in nature and the molecules are believed to function as an evolutionarily conserved chemical language used by nematodes to control many aspects of their development. Ascarosides are also perceived by other organisms and have been demonstrated to have a range of effects on numerous organisms including: bacteria, fungi, plants, and mammals including humans.

Ascarosides are derivatives of the sugar ascarylose-a di-deoxy sugar lacking hydroxyl groups at the 3- and 6-positions. Ascarosides have the general structure shown in Formula I:

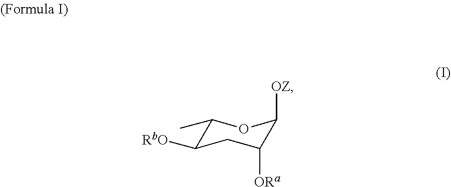

wherein:
Z is an optionally substituted C₂₋₄₀ aliphatic group, and
each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g. —a moiety —C(O)OR^{c}), a C₂₋₂₀ carbamate (e.g. —a moiety —C(O)N(R^{c})₂), a C₂₋₂₀ thioester (e.g. a moiety —C(S)R^{c}), a C₂₋₂₀ thiocarbonate (e.g. a moiety —C(S)OR^{c}), a C₂₋₂₀ dithiocarbonate (e.g. a moiety —C(S)SR^{c}), a C₁₋₂₀ thiocarbamate (e.g. a moiety —C(S)N(R^{c})₂), a sugar moiety, a peptide, a polymer chain, or a linkage via a bond or a carbon-containing linker moiety to another ascaroside molecule. Where R^{c} is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule, and where R^{a} and R^{b} may be taken together to form an optionally substituted ring, optionally containing one or more heteroatoms, and optionally containing one or more sites of unsaturation.

In certain embodiments, Z is:
(i) —CH(CH₃)—R¹, where R¹ is an optionally substituted C₁₋₄₀ aliphatic group
(ii) —CH(CH₃)—(CH₂)ₙ—CO₂R², where n is an integer 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(iii) —CH(CH₃)—(CH₂)ₙ—CH═CH—CO₂R², where n is an integer 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(iv) —CH(CH₃)—(CH₂)ₙ—CH(OH)—CH—CO₂R³², where n is an integer 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(v) —CH(CH₃)—(CH₂)ₙ—C(O)—CH—CO₂R², where n is an integer 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(vi) —(CH₂)ₙ—CO₂R², where n is an integer 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(vii) —(CH₂)ₙ—CH═CH—CO₂R², where n is an integer 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(viii) —(CH₂)ₙ—CH(OH)—CH—CO₂R², where n is an integer 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule; or
(ix) —(CH₂)ₙ—C(O)—CH—CO₂R², where n is an integer 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule.

In certain embodiments, Z is:
(x) —CH(CH₃)—(CH₂)ₙ—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xi) —CH(CH₃)—(CH₂)ₙ—CH═CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xii) —CH(CH₃)—(CH₂)ₙ—CH(OH)—CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xiii) —CH(CH₃)—(CH₂)ₙ—C(O)—CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xiv) —(CH₂)ₙ—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xv) —(CH₂)ₙ—CH═CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xvi) —(CH₂)ₙ—CH(OH)—CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule; or
(xvii) —(CH₂)ₙ—C(O)—CH—CON(R³)₂, where n is an integer 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule.

In certain embodiments, R^{a} is —H.

In certain embodiments R^{b} is —H.

In certain embodiments, R^{a} and R^{b} are the same. In certain embodiments R^{a} and R^{b} are both —H.

In certain embodiments, R^{a} and R^{b} are different. In certain embodiments, R^{a} is —H, and R^{b} is other than —H. In certain embodiments, R^{a} is other than —H and R^{b} is —H. In certain embodiments, R^{a} is —H and R^{b} is p-hydroxybenzoate. In certain embodiments, R^{a} is —H and R^{b} is indole-3-carboxylate. In certain embodiments, R^{a} is —H and R^{b} is (E)-2-methyl-2-butenoate. In certain embodiments, R^{a} is —H and R^{b} is picolinate. In certain embodiments, R^{a} is —H and R^{b} is nicotinate. In certain embodiments, R^{a} is —H and R^{b} is (R)-2-hydroxy-2-(4-hydroxyphenyl) ethyl) amino)-4-oxobutanoate. In certain embodiments, R^{a} is —H and R^{b} is 4-((4-hydroxyphenethyl) amino)-4-oxobutanoate.

In certain embodiments R^{a} and R^{b} are both —H, and Z is selected from the formulae defined in (i) to (ix) above. In certain embodiments R^{a} and R^{b} are both —H, and Z conforms to formula (i) above. In certain embodiments R^{a} and R^{b} are both —H, and Z conforms to formula (ii) above. In certain embodiments R^{a} and R^{b} are both —H, and Z conforms to formula (iii) above. In certain embodiments R^{a} and R^{b} are both —H, and Z conforms to formula (iv) above. In certain embodiments R^{a} and R^{b} are both —H, and Z conforms to formula (v) above. In certain embodiments R^{a} and R^{b} are both —H, and Z conforms to formula (vi) above. In certain embodiments R^{a} and R^{b} are both —H, and Z conforms to formula (vii) above. In certain embodiments R^{a} and R^{b} are both —H, and Z conforms to formula (viii) above. In certain embodiments R^{a} and R^{b} are both —H, and Z conforms to formula (ix) above. In certain embodiments R^{a} and R^{b} are both —H, and Z conforms to formula (x) above. In certain embodiments R^{a} and R^{b} are both —H, and Z conforms to formula (xi) above. In certain embodiments R^{a} and R^{b} are both —H, and Z conforms to formula (xii) above. In certain embodiments R^{a} and R^{b} are both —H, and Z conforms to formula (xiii) above. In certain embodiments R^{a} and R^{b} are both —H, and Z conforms to formula (xiv) above. In certain embodiments R^{a} and R^{b} are both —H, and Z conforms to formula (xv) above. In certain embodiments R^{a} and R^{b} are both —H, and Z conforms to formula (xvi) above. In certain embodiments R^{a} and R^{b} are both-H, and Z conforms to formula (xvii) above.

In certain embodiments, R² is —H. In certain embodiments, R² is a metal cation. In certain embodiments, R² is an organic cation (e.g. a nitrogen or phosphorous centered cationic group). In certain embodiments, R² is an optionally substituted C₁₋₂₀ aliphatic group. In certain embodiments, R² is an optionally substituted C₁₋₁₂ aliphatic group. In certain embodiments, R² is an optionally substituted C₁₋₈aliphatic group. In certain embodiments, R² is an optionally substituted C₁₋₆ aliphatic group. In certain embodiments, R² is selected from methyl, ethyl, n-propyl, i-propyl, n-butyl, sec-butyl, and t-butyl. In certain embodiments, R² is an optionally substituted aromatic group. In certain embodiments, R² is a glycoside. In certain embodiments, R² comprises an amino acid. In certain embodiments, R² comprises a peptide. In certain embodiments, R² comprises a nucleotide.

In certain embodiments, at least one R³ is —H. In certain embodiments, both R³ groups are —H. In certain embodiments, at least one R³ is an optionally substituted C₁₋₂₀ aliphatic group. In certain embodiments, both R³ groups are an optionally substituted C₁₋₂₀ aliphatic group which may be the same or different. In certain embodiments, at least one R³ is an optionally substituted C₁₋₁₂ aliphatic group. In certain embodiments, at least one R³ is an optionally substituted C₁₋₈ aliphatic group. In certain embodiments, at least one R³ is an optionally substituted C₁₋₆ aliphatic group. In certain embodiments, at least one R³ is selected from methyl, ethyl, n-propyl, i-propyl, n-butyl, sec-butyl, and t-butyl. In certain embodiments, at least one R³ is —CH₂CH₂OH. In certain embodiments, at least one R³ is —CH₂CH₂OR². where R² is as defined in the genera and subgenera herein. In certain embodiments, at least one R³ is an optionally substituted aromatic group. In certain embodiments, at least one R³ comprises a glycoside. In certain embodiments, at least one R³ comprises an amino acid. In certain embodiments, at least one R³ at least one R³ comprises a peptide. In certain embodiments, at least one R³ comprises a nucleotide.

In certain embodiments, an ascaroside is selected from the group consisting of:

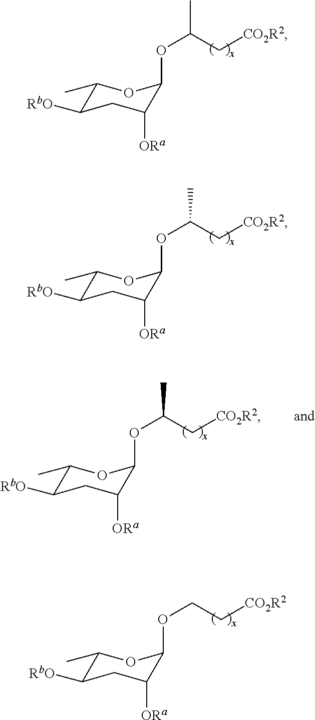

where x is an integer from 1 to 22, and each of R^{a}, R^{b}, and R² is as defined above and in the genera and subgenera herein.

In certain embodiments, an ascaroside is selected from the group consisting of:

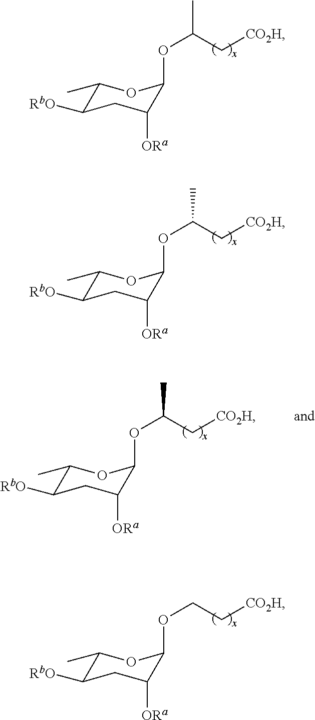

where each of x, R^{a}, and R^{b}, is as defined above and in the genera and subgenera herein.

In certain embodiments, an ascaroside is selected from the group consisting of:

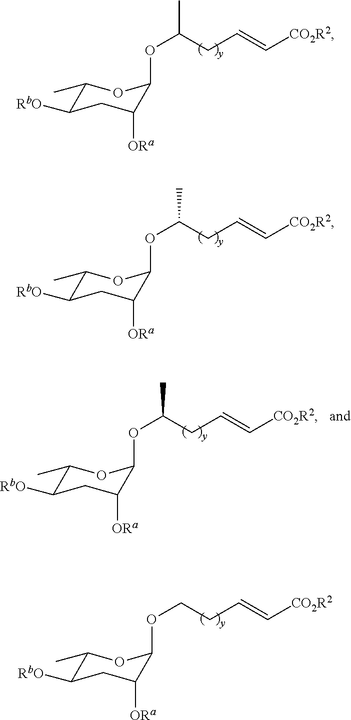

where y is an integer from 1 to 20, and each of R^{a}, R^{b}, and R² is as defined above and in the genera and subgenera herein.

In certain embodiments, an ascaroside is selected from the group consisting of:

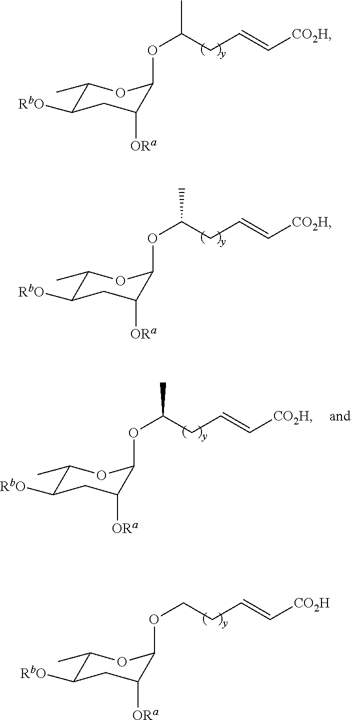

where each of y, R^{a}, and R^{b}, is as defined above and in the genera and subgenera herein.

In certain embodiments, an ascaroside is selected from the group consisting of:

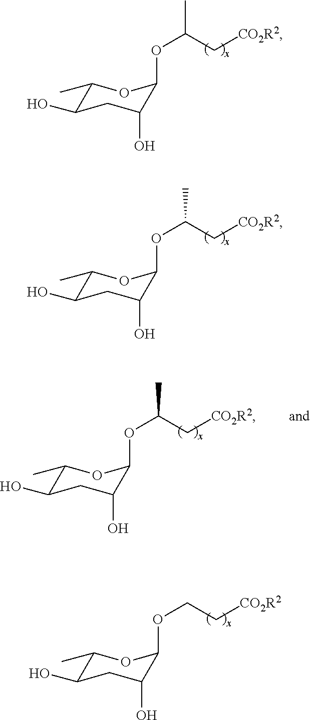

where x is an integer from 1 to 22, and R² is as defined above and in the genera and subgenera herein.

In certain embodiments, an ascaroside is selected from the group consisting of:

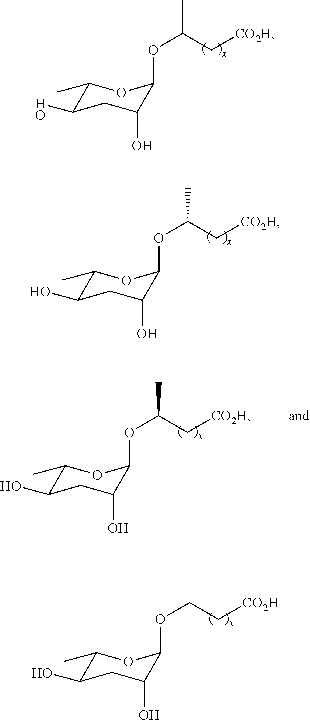

where x is as defined above and in the genera and subgenera herein.

In certain embodiments, an ascaroside is selected from the group consisting of:

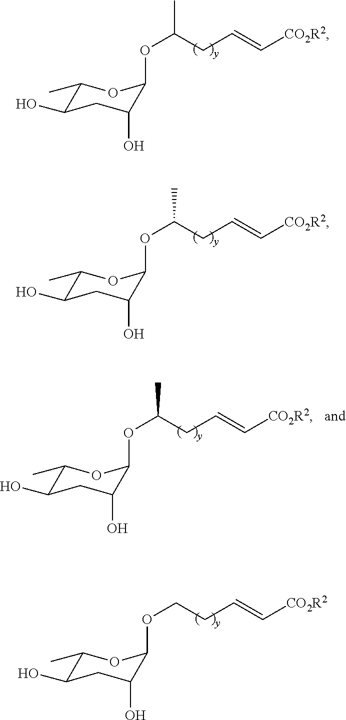

where y is an integer from 1 to 20, and R² is as defined above and in the genera and subgenera herein.

In certain embodiments, an ascaroside is selected from the group consisting of:

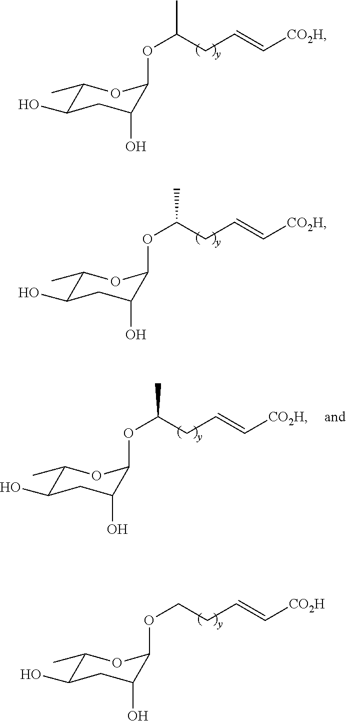

where y is as defined above and in the genera and subgenera herein.

In certain embodiments, an ascaroside is selected from the group consisting of:

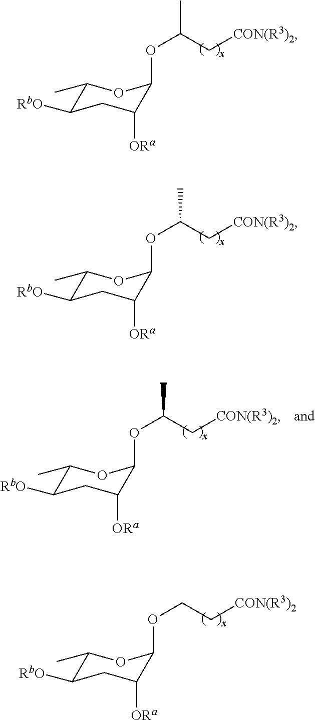

where x is an integer from 1 to 22, and each of R^{a}, R^{b}, and R³ is as defined above and in the genera and subgenera herein.

In certain embodiments, an ascaroside is selected from the group consisting of:

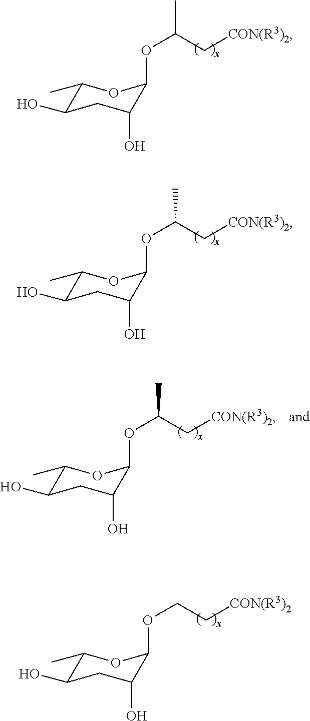

where each of x and R³ is as defined above and in the genera and subgenera herein.

In certain embodiments, an ascaroside is selected from the group consisting of:

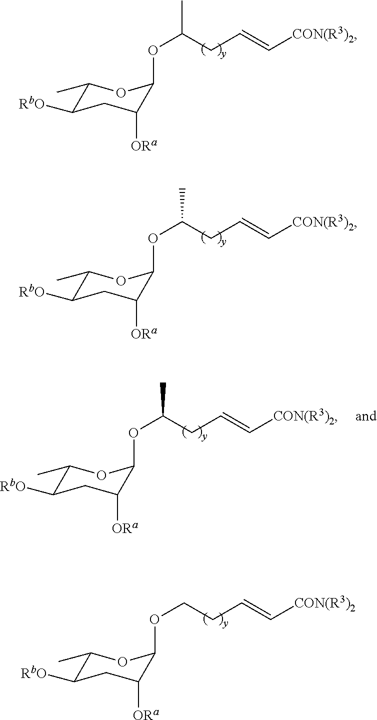

where y is an integer from 1 to 20, and each of R^{a}, R^{b}, and R² is as defined above and in the genera and subgenera herein.

In certain embodiments, an ascaroside is selected from the group consisting of:

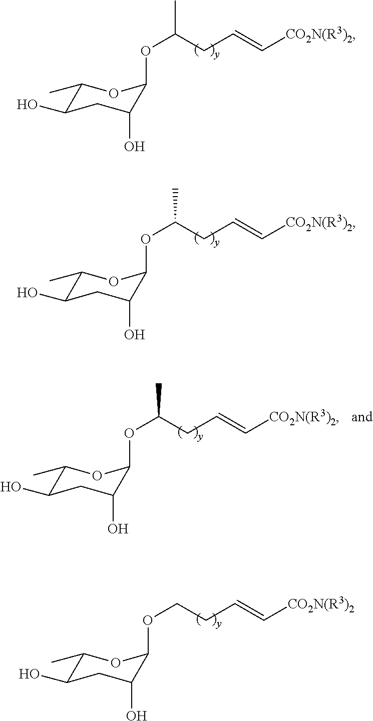

where each of y and R³ is as defined above and in the genera and subgenera herein.

In certain embodiments, x in any of the structures herein is an integer between 2 and 18 inclusive. In certain embodiments, x in any of the structures herein is an integer between 4 and 10 inclusive. In certain embodiments, x in any of the structures herein is an integer between 6 and 12 inclusive. In certain embodiments, x in any of the structures herein is an integer between 5 and 9 inclusive. In certain embodiments, x in any of the structures herein is 7, 8 or 9. In certain embodiments, x in any of the structures herein is 6. In certain embodiments, x in any of the structures herein is 7. In certain embodiments, x in any of the structures herein is 8. In certain embodiments, x in any of the structures herein is 9. In certain embodiments, x in any of the structures herein is 10. In certain embodiments, x in any of the structures herein is 11. In certain embodiments, x in any of the structures herein is 12.

In certain embodiments, x in any of the structures herein is an integer greater than 5. In certain embodiments, x in any of the structures herein is an integer greater than 6, greater than 7, greater than 9, or greater than 10. In certain embodiments, x in any of the structures herein is an integer between 10 and 20.

In certain embodiments, provided modified ascarosides can comprise a mixture of ascarosides varying only in the value of x (i.e., the modified ascaroside comprises a mixture of congeners with varying sidechain lengths). In certain embodiments, such modified ascarosides are characterized in that the average value of x in the modified ascaroside is between about 2 and about 20). In certain embodiments, such modified ascarosides are characterized in that the average value of x in the composition is between about 3 and about 10, between about 4 and about 12, between about 6 and about 10, or between about 7 and about 9. In certain embodiments, such modified ascarosides are characterized in that the average value of x is greater than about 4, greater than about 5, greater than about 6, greater than about 7, or greater than about 8. The average value of x in such modified ascarosides can be determined by various methods well known in the art including, but not limited to analyzing the mixture by ¹H NMR spectroscopy, mass spectroscopy, high performance liquid chromatography and the like.

In certain embodiments, y in any of the structures herein is an integer between 2 and 18 inclusive. In certain embodiments, y in any of the structures herein is an integer between 4 and 10 inclusive. In certain embodiments, y in any of the structures herein is an integer between 6 and 12 inclusive. In certain embodiments, y in any of the structures herein is an integer between 5 and 9 inclusive. In certain embodiments, y in any of the structures herein is 7, 8 or 9. In certain embodiments, y in any of the structures herein is 6. In certain embodiments, y in any of the structures herein is 7. In certain embodiments, y in any of the structures herein is 8. In certain embodiments, y in any of the structures herein is 9. In certain embodiments, y in any of the structures herein is 10. In certain embodiments, y in any of the structures herein is 11. In certain embodiments, y in any of the structures herein is 12.

In certain embodiments, y in any of the structures herein is an integer greater than 5. In certain embodiments, y in any of the structures herein is an integer greater than 6, greater than 7, greater than 9, or greater than 10. In certain embodiments, y in any of the structures herein is an integer between 10 and 20.

In certain embodiments, provided modified ascarosides can comprise a mixture of ascarosides varying only in the value of y (i.e., the modified ascaroside comprises a mixture of congeners with varying sidechain lengths). In certain embodiments, such modified ascarosdies are characterized in that the average value of y is between about 2 and about 20. In certain embodiments, such modified ascarosides are characterized in that the average value of y in the composition is between about 3 and about 10, between about 4 and about 12, between about 6 and about 10, or between about 7 and about 9. In certain embodiments, such modified ascarosides are characterized in that the average value of y is greater than about 4, greater than about 5, greater than about 6, greater than about 7, or greater than about 8. The average value of y in such modified ascarosides can be determined by various methods well known in the art including, but not limited to analyzing the mixture by ¹H NMR spectroscopy, mass spectroscopy, high performance liquid chromatography and the like.

In an embodiment, ascarosides useful for the present invention have the general structure (I), where Z is —CH(CH₃)—(CH₂)ₙ—CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide.

In an embodiment, ascarosides useful for the present invention have the general structure (I) where Z is —CH(CH₃)—(CH₂)ₙ—CH═CH-CO₂R², where n is an integer 1 to 40, and R² is —H, a metal cation, an optionally substituted C₁₋₂₀ aliphatic group, an optionally substituted aromatic group, a glycoside, an amino acid, a peptide, or a nucleotide.

Specific ascarosides that are useful for the present invention include, but are not limited to, ascr#7 and ascr#18.

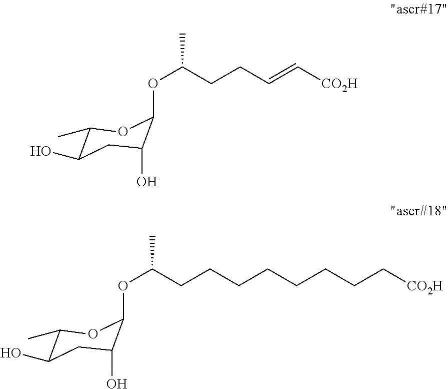 [SMILES: CC1OC(O[C@H](C)CC/C=C/C(=O)O)C(O)CC1O ; CC1OC(O[C@H](C)CCCCCCCCC(=O)O)C(O)CC1O]

In certain embodiments, an ascaroside used in the provided methods is selected from the group consisting of: ascr#9, ascr#12, ascr#14, ascr#1, ascr#10, ascr#16, ascr#18, ascr#20, ascr#22, ascr#24, ascr#26, ascr#28, ascr#30, ascr#32, ascr#34, and ascr#36. In certain embodiments, an ascaroside used in the provided methods is selected from the group consisting of: ascr#10, ascr#16, ascr#18, ascr#20, ascr#22, and ascr#24. In certain embodiments, an ascaroside used in the provided methods is selected from the group consisting of: ascr#9, ascr#14, ascr#10, and ascr#18.

In certain embodiments, an ascaroside used in the provided methods is selected from the group consisting of: ascr#5, oscr#9, oscr#12, oscr#1, oscr#14, oscr#10, oscr#16, oscr#18, oscr#20, oscr#22, oscr#24, oscr#26, oscr#28, oscr#30, oscr#32, oscr#34, and oscr#36. In certain embodiments, an ascaroside used in the provided methods is selected from the group consisting of: oscr#10, oscr#16, oscr#18, oscr#20, and oscr#22. In certain embodiments, an ascaroside used in the provided methods is selected from the group consisting of: bhas#5, oscr#9, oscr#12, oscr#1, oscr#14, oscr#10, oscr#16, oscr#18, oscr#20, oscr#22, oscr#24, oscr#26, oscr#28, oscr#30, oscr#32, oscr#34, and oscr#36. In certain embodiments, an ascaroside used in the provided methods is selected from the group consisting of: oscr#10, oscr#16, oscr#18, oscr#20, and oscr#22.

In certain embodiments, an ascaroside used in the provided methods is selected from the group consisting of: bhas#9, bhas#10, bhas#16, bhas#18, bhas#22, bhas#24, bhas#26, bhas#28, bhas#30, bhas#32, bhas#34, bhas#36, bhas#38, bhas#40, and bhas#42.

In certain embodiments, an ascaroside used in the provided methods is selected from the group consisting of: bhos#10, bhos#16, bhos#18, bhos#22, bhos#24, bhos#26, bhos#28, bhos#30, bhos#32, bhos#34, bhos#36, bhos#38, bhos#40, and bhos#42.

In certain embodiments, an ascaroside used in the provided methods is selected from the group consisting of: ascr#18, oscr#16, oscr#17, oscr#15, bhas#18, bhos#16, glas#18, dhas#18, ibha#18, ibho#16, icas#18, icos#15, icos#16, and any combination of two or more of these.

Ascarosides can be obtained from natural sources (e.g., nematodes) or they may be prepared synthetically. Ascarosides can be prepared synthetically, for example, by converting 1-O-substituted rhamnose to 1-O-substituted ascarylose. An exemplary method of preparing ascarosides includes: providing as a feedstock a 1-O-substituted rhamnose; forming a mono-sulfonate ester at the 3-OH group of the feedstock; and treating the mono-sulfonate ester with a hydride source to form a 1-O-substituted ascarylose. In certain embodiments, forming the mono-sulfonate ester is conducted on a substrate without hydroxyl protecting groups at the 2- or 4-position of the rhamnose feedstock. In certain embodiments, such methods comprise contacting the feedstock with a sulfonating agent (i.e., a sulfonyl halide, sulfonic anhydride or similar reagent) in the presence of a Lewis acid. Specific details regarding the synthesis of 1-O-substituted ascarylose can be found in PCT Application No. PCT/IB2021/056981, which is incorporated herein by reference.

### Structurally Modified Ascarosides

As referenced above, in some embodiments of the present disclosure, an ascaroside molecule is modified so as to provide a compound that results in modified release of the ascaroside molecule during use (e.g., when applied to a plant, to an animal, or to the environment around a plant or animal). Some such structurally modified ascarosides comprise an oligomer or polymer at least partially composed of ascarosides. In some embodiments, the structural modification is generally such that the modification is reversible, i.e., the structurally modified ascaroside may release one or more ascaroside molecules upon one or more environmental exposures/triggers or upon degradation.

In some embodiments, the structurally modified ascaroside is an ascaroside-based oligomer or polymer, which comprises an oligomer (e.g., dimer, trimer, tetramer and the like) or a polymer of an ascaroside (i.e., comprising ascaroside-containing repeat units). The oligomer or polymer can comprise two or more connected ascarosides of the same formula and/or can comprise two or more connected ascarosides of different formulae. In some embodiments, ascaroside molecules are connected directly to one another via covalent linkages between functional groups already present on the ascaroside molecules. For example, in some embodiments, such structurally modified ascarosides can comprise ester linkages that can be formed by condensation of a carboxylic acid on the side chain of one ascaroside (e.g., present within certain embodiments as a component of the “Z” group in Formula (I) above) with a hydroxyl functional group on another ascaroside molecule (e.g., wherein Rₐ or R_{b} of Formula (I) is H or where the ascaroside otherwise comprises an OH substituent (for example wherein the Z group of Formula (I) comprises an OH substituent)). In some embodiments, ascaroside molecules are connected to one another indirectly via covalent linkages between a multivalent linker and functional groups present on the ascaroside molecules.

In some embodiments, such ascaroside-based oligomers or polymers comprise oligomers or polymers consisting substantially of ascaroside molecules (e.g. oligomers or polymers where ascarosides comprise the primary monomer from which the oligomer or polymer is composed). In other embodiments, such compositions comprise co-polymers or co-oligomers of ascaroside molecules with multivalent linkers or other monomers.

In certain embodiments, provided ascaroside based oligomers or polymers conform to Formula (II):

A-[(L)ₘ-A]ₚ or L-[A-(L)ₘ]ₚ (Formula II),

wherein each A is independently an ascaroside molecule; L is a multivalent linker moiety covalently bound to A, m is 0 or 1, and p is an integer between 1 and 1000. In some embodiments, the oligomers or polymers comprise a covalent linkage to a carboxylic acid functional group on A (e.g., via an ester or amide bond). In some embodiments, the oligomers or polymers comprise a covalent linkage to a hydroxyl functional group on A (e.g., via an ether, ester, glycosidic, urethane, carbonate, or siloxane linkage). As such, the composition of the optional multivalent linker L can vary widely.

In certain embodiments, provided ascaroside based oligomers or polymers conform to Formula (III):

[(L)ₘ-A]ₚ or [A-(L)ₘ]ₚ (Formula III),

wherein each A is independently an ascaroside molecule; L is a multivalent linker moiety covalently bound to A, m is 0 or 1, and p is an integer between 1 and 1000. In some embodiments, the oligomers or polymers comprise a covalent linkage to a carboxylic acid functional group on A (e.g., via an ester or amide bond). In some embodiments, the oligomers or polymers comprise a covalent linkage to a hydroxyl functional group on A (e.g., via an ether, ester, glycosidic, urethane, carbonate, or siloxane linkage). As such, the composition of the optional multivalent linker L can vary widely.

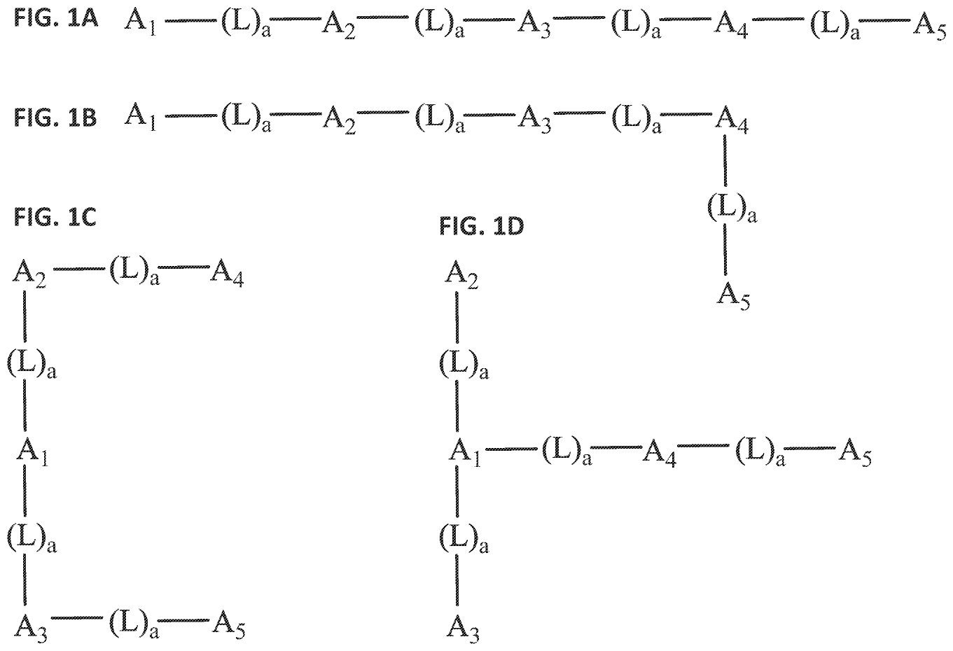

Such ascaroside-based oligomers and polymers provided according to the present disclosure can be linear or branched. Certain, non-limiting examples of oligomers containing five linked ascaroside (“A”) moieties are illustrated in FIG. 1A, wherein each of A₁, A₂, A₃, A₄, and A₅ and all “L” groups can be the same or different. Each “a” is an integer that is 0 or 1, i.e., each A may be directly linked to an adjacent A via a bond or can include a linker (L) therebetween. It is to be understood that, for modified ascarosides in the form of further oligomers and polymers, each connection between ascaroside moieties can result in the formation of a linear or branched section of the respective oligomer or polymer depending, e.g., on the point of connection between the ascaroside moiety and an adjacent ascaroside moiety or between the ascaroside moiety and L.

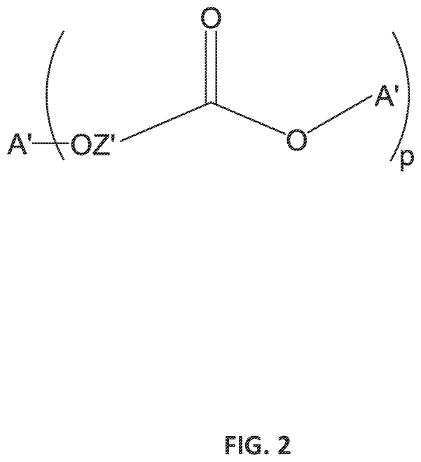

In some embodiments, such ascaroside-based oligomers or polymers can comprise ester linkages between adjacent ascaroside moieties. A non-limiting representative structure of an oligomeric or polymeric modified ascaroside comprising such ester linkages is illustrated in FIG. 2, wherein A′ refers to the portions of ascaroside molecules not explicitly represented in the figure (i.e., including the tetrahydropyranyl ring and at least a portion (or all) of the OR^{a}-, OR^{b}-, and/or OZ substituents), and where each A′ can be the same or different, and wherein p is an integer from 1 to 1000. Oligomers and polymers within the scope of the disclosure can be linear (e.g., with all A′-containing subunits are connected to at most two adjacent subunits in) or can be branched (e.g., where at least some A′-containing subunits are connected directly or via linker to more than two adjacent A subunits). In some embodiments, with the exception of the terminal A′ subunits, each A′ is connected to two other A′ subunits via ester linkages. In some embodiments, with the exception of the terminal A′ subunits, at least some A′ subunits are connected to three other A′ subunits.

Chemical reactions that can suitably be employed to produce oligomers and polymers from molecules containing hydroxyl groups and/or carboxylic acids are well known in the art, and can be readily adapted to provide oligomers or polymers comprising ascarosides. For example, catalysts, methods, and processing techniques that can be employed are described in Textbook of Polymer Science, 3rd Edition by Fred W. Billmeyer (ISBN: 978-0-471-03196-3), which is incorporated herein by reference in its entirety. Depending on the oligomeric or polymeric structure targeted, it may be desirable to utilize ascaroside precursors having one or more reactive functional groups masked with a protecting group. In this approach, covalent bonds can be formed to a specific functional group on the ascaroside molecules without participation or interference of other functional groups. Once the desired oligomer or polymer has been formed, such protecting groups can be removed. Suitable protecting groups and strategies for their formation and removal are described in Greene's Protective Groups in Organic Synthesis by Peter G. M. Wuts and Theodora W. Greene (ISBN: 9780471697541), which is incorporated herein by reference in its entirety. In certain embodiments, an ascaroside precursor having one or more of the hydroxyl groups on the ascarylose sugar protected is oligomerized via reaction of a carboxylic acid group (or a reactive derivative thereof) present on the ascaroside sidechain. In certain embodiments, an ascaroside precursor having a protecting group masking the reactivity of a carboxylic acid group present on the ascaroside sidechain is oligomerized via reaction of one or more hydroxyl groups present on the ascarylose sugar.

In certain embodiments, ascarylose oligomers or polymers are formed by first reacting an ascarylose or a derivative thereof with a reagent that attaches a polymerizable group into the molecule—for example, a polymerizable functional group such as an olefin, an amine, an isocyanate, an epoxide, an acyl halide, or the like for which known polymerization processes exist. Such polymerizable functional groups can be introduced by means well known in the art of synthetic organic chemistry. Methods to incorporate polymerizable functional groups onto the ascaroside may require the use of protecting groups as described above to ensure the group is attached at a desired position or that other functional groups present on the ascaroside do not interfere with the process of attaching the functional group or with subsequent processes necessary to form the oligomers or polymers. Such polymerizable functional groups are preferably attached to the ascaroside substrate through bonds that are labile under biotic or environmental conditions such that when the resulting oligomer or polymers degrade, free ascaroside molecules can be liberated.

In certain embodiments, the multivalent linker “L” may comprise an oligomeric or polymeric group (e.g., L may consist of a chain of repeating monomer subunits). In certain embodiments, L comprises a polyester, a polyamide, a polyether, a polycarbonate, or a polyolefin. In certain embodiments, L comprises a biopolymer. In certain embodiments, L comprises a carbohydrate, a peptide, a protein, RNA, or DNA. In certain embodiments, L comprises a carbohydrate. In certain embodiments, L comprises starch, cellulose, chitosan, or chitin. In certain embodiments, L comprises a peptide or a protein. In certain embodiments, L comprises a polynucleotide.

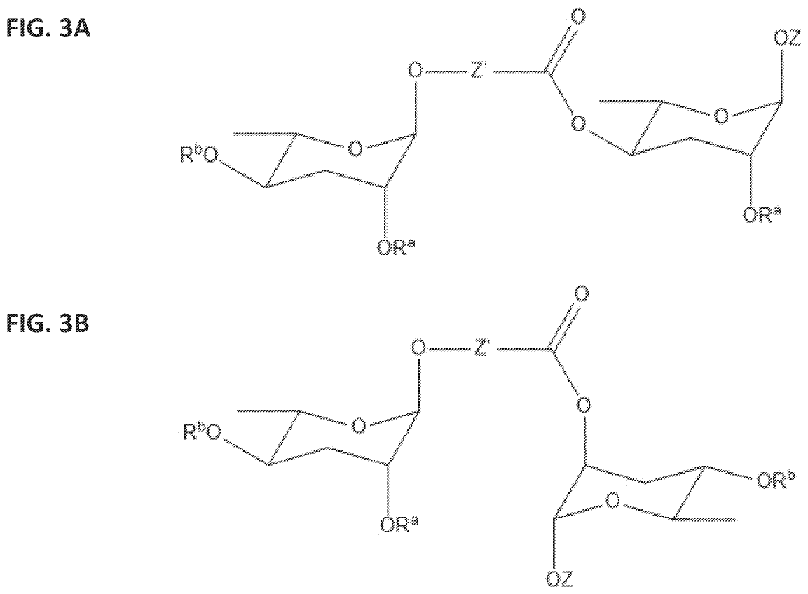

Non-limiting examples of certain specific ascaroside dimers within the scope of the disclosure are provided in FIGS. 3A and 3B (where a=0, i.e., no “L” linker group is present between the ascaroside moieties). One of skill in the art will appreciate that each ascaroside can be connected to an adjacent ascaroside via its OZ group (condensed with the OR^{a} or OR^{b} substituent on an adjacent ascaroside) or via its OR^{a} or OR^{b} group (esterified with a carboxyl moiety in the OZ substituent on an adjacent ascaroside). In certain embodiments, an oligomer is provided wherein each ascaroside is connected in the same manner (e.g., the Z group on a first ascaroside is esterified with the R^{a} group on a second ascaroside, the Z group on the second ascaroside is esterified with the R^{a} group on a third ascaroside, the Z group on the third ascaroside is esterified with the R^{a} group on a fourth ascaroside, and so on). In other embodiments, an oligomer is provided wherein each ascaroside is randomly connected (e.g., the Z group on a first ascaroside is esterified with the R^{a} group on a second ascaroside, the Z group on the second ascaroside is esterified with the R^{b} group on a third ascaroside, and the R^{a} group of the third ascaroside is esterified with a Z group on a fourth ascaroside.

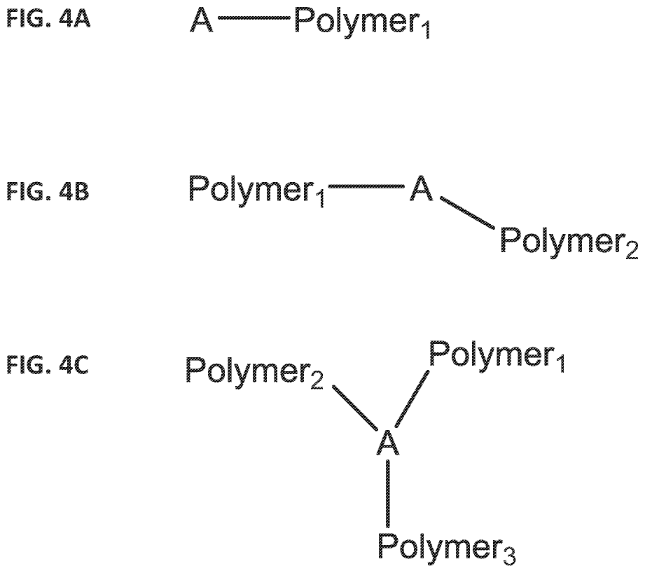

In some embodiments, a structurally modified ascaroside as provided herein is an oligomer-functionalized ascaroside or a polymer-functionalized ascaroside, which comprises an ascaroside chemically bonded to a natural or synthetic oligomer or polymer. For example, any one or more of Z, R^{a}, and R^{b} on the ascaroside of Formula (I) above can be functionalized with an oligomer or polymer. Three example embodiments are depicted in FIG. 4, where each of Polymer₁, Polymer₂, and Polymers can be the same or different in composition and/or length and where each of the referenced polymers can be present or absent in different embodiments (e.g., the molecule can comprise only one polymer attached to the ascaroside, the molecule can comprise two polymers attached to different moieties on the ascaroside, or the molecule can comprise three polymers attached to different moieties on the ascaroside. The polymer(s) can be covalently bonded to the ascaroside in a manner similar to that described above with respect to ascaroside-based oligomers or polymers. Specifically, considering the ascaroside structure of Formula (I), pendant polymers can be incorporated on one, two, or all of the Z, ORₐ, or OR_{b} substituents to give a polymer-functionalized ascaroside. The polymer(s) on the polymer-functionalized ascaroside may be linear polymers, branched polymers, hyperbranched polymers, bottle-brush polymers, or dendritic polymers. Polymers can comprise a single repeat unit or can be copolymers (e.g., block copolymers, random copolymers, graft copolymers, tapered copolymers or alternating copolymers). The number of repeat units/length of the oligomer or polymer can vary; in some embodiments, the oligomer or polymer comprises from about two to about one thousand repeat units. Polymers can be natural or synthetic or can be a hybrid thereof.

Suitable polymers include all agriculturally acceptable polymers that can be suitably functionalized so as to be covalently bound to a moiety present on the ascaroside molecule. In preferred embodiments, such polymers are biodegradable polymers. In some embodiments, such polymers are bio-based or natural polymers. In some embodiments, such polymers are synthetic polymers. Certain, non-limiting examples of suitable polymers include: chitosan, chitin, starches, lignin, agar, pectin, cellulose, other polysaccharides (e.g., bark, sawdust, other cellulosic waste), poly(ethylene glycol), poly(caprolactone), poly(lactide), poly(glycolic acid), polybutylene, poly(vinyl alcohol), poly(vinyl chloride), poly(citric acid), poly(acrylate), poly(aspartic acid), poly(ethylene), poly(ethylene vinyl-acetate), polyethylene vinyl acetate (PEVA), polystyrene, poly divinylbenzene, and derivatives and copolymers thereof.

Suitable polymers include pharmaceutically acceptable polymers. Such pharmaceutically acceptable polymers can be suitably functionalized so as to be covalently bound to a moiety present on the ascaroside molecule. In preferred embodiments, such polymers arc biocompatible polymers and/or metabolically labile polymers.

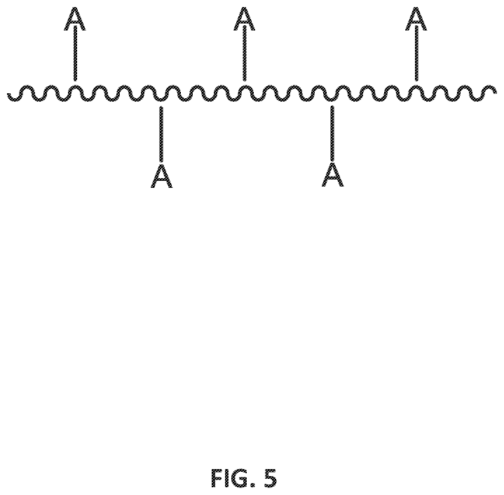

In further embodiments, a structurally modified ascaroside is an ascaroside-pendant polymer, where one or more ascarosides are bonded directly or indirectly to the backbone of a polymer as pendant groups thereon, e.g., as shown in FIG. 5. It is noted that, in FIG. 5, for simplicity, the polymer is shown as having five pendant ascaroside (“A”) groups; however, it is to be understood that the disclosure is not so limited; the number of pendant A groups on a given polymer chain can range in some embodiments, e.g., from 2 to about 1,000. The composition of the polymer is widely variable and can be, e.g., any of the agriculturally acceptable polymers referenced herein above. In certain embodiments, the composition comprises a polymer such as polyvinyl alcohol or polyvinyl acetate having pendant hydroxy or acyloxy groups that can be esterified or transesterified with a carboxyl group of an ascaroside or a modified ascaroside. In certain embodiments, the composition comprises a polymer such as polyvinyl alcohol, polyvinyl chloride or polyvinyl acetate having pendant functional groups such as halides, alcohols, or acyloxy groups that can form ether linkages with hydroxyl groups on an ascaroside or ascaroside derivative. In certain embodiments, the composition comprises a polymer such as polyacrylic acid or a polyacrylate having pendant carboxy or carboxyester groups that can be esterified or transesterified with a hydroxyl group on an ascaroside or a modified ascaroside or can form anhydride linkages with a carboxyl group on an ascaroside.

In some embodiments, a structurally modified ascaroside as provided herein is an oligomer-functionalized ascaroside or a polymer-functionalized ascaroside, which comprises an ascaroside bound to a naturally-occurring oligomer, polymer, or biomolecule. In some embodiments, a structurally modified ascaroside comprises an ascaroside covalently bound to starch, cellulose, lignin, or similar biopolymeric materials.

In some embodiments of the ascaroside-pendant polymer, pendant ascaroside molecules are attached to the polymer via non-covalent interactions, e.g., hydrogen bonding and/or ionic bonding. In some embodiments, ascaroside molecules are covalently linked to the polymer directly or via various types of linkages, which are advantageously cleavable via an environmental trigger such that that under certain conditions, the linkages are cleaved, releasing the ascaroside or a derivative thereof. Examples of non-covalent linkages include mixtures of polycationic polymers or oligomers with the carboxylate anions of ascarosides-for example, derived from ascarosides having a carboxylic acid moiety on the sidechain or from ascarosides that have been modified to provide such a carboxylate group (i.e., via reaction of an ascaroside hydroxyl group with a cyclic anhydride such as succinic-, maleic-, or phthalic anhydride). Examples of suitable covalent linkages include, but are not limited to, ether, ester, amide, glycosidic, urethane, carbonate, or siloxane linkages. Examples of particularly suitable covalent linkages include ester, amide and glycosidic linkages.

The rate of release of ascaroside moieties from such structurally modified ascarosides may, in some embodiments, be dependent upon the nature of the respective polymer properties, e.g., hydrophilic/hydrophobic ratio, linear/branched nature, polymer length, degree of cross-linking, degree of non-covalent interactions (e.g., hydrogen bonding and/or ionic interaction), and type(s) of bonds within the structure. In some embodiments, the release properties are dependent at least in part on the nature of the bond(s) by which the ascaroside(s) are linked to the remainder of the molecule; in some embodiments, the ascaroside can be released by hydrolysis, thermodynamic dissociation, microbial degradation or another chemical reaction that breaks the linkage(s) to the ascaroside. Release properties can be effectively tuned by modifying these properties.

### Physically Modified Ascarosides

In some embodiments, the release of ascarosides is modified via physical methods, e.g., via incorporation of the ascarosides within a matrix designed for modified release thereof. In some such embodiments, release of the ascaroside from the matrix can be, e.g., based on diffusion and/or matrix degradation. It is noted that although such embodiments are referenced herein as comprising “physical” modification, such modifications do not preclude the presence of structural modifications, i.e., some of the methods provided below may result in changes to the ascaroside such that the ascaroside structure may, in fact, be modified to some extent (e.g., via introducing bonds (covalent or ionic) to one or more other components).

In certain embodiments, a physically modified ascaroside is provided by mixing or compounding one or more ascarosides with a polymeric material (“organic compounding”) and/or an inorganic material (“inorganic compounding”). In some embodiments, nanocomposites comprising an ascaroside compounded with both a polymeric material and an inorganic material are provided.

In some embodiments, an organic compounded ascaroside is provided. Organic compounding may involve mixing and/or blending of an organic component (e.g., a polymeric resin) and one or more additives (here, the one or more ascarosides and, optionally, one or more additional components, such as the types of agronomically acceptable excipients and additives referenced herein below). In some embodiments, such embodiments can be described as formulations wherein the ascaroside is impregnated within the organic component. Compounding in some embodiments can involve heating (e.g., such that the organic component (e.g., polymeric resin) is in melted form during the mixing and/or blending). In some embodiments, the compounding is done during an extrusion process as known in the art, such that the resulting extrudate (which can be of varying sizes and shapes) comprises the one or more ascarosides.

In some such embodiments, the compounding comprises formation of covalent bonds between the organic (e.g., polymeric) material and at least one of the one or more ascarosides. In other embodiments, substantially no covalent bonds are formed between the organic (e.g., polymeric) material and the ascaroside(s).

In certain embodiments, ascaroside compositions that are compounded with polymers include compositions resulting from reactive extrusion of polymers with one or more ascarosides. For example, such compositions may comprise compositions resulting from reactive extrusion of polyesters (particularly biodegradable polyesters) with ascarosides. Such reactive extrusion can result in partial or complete incorporation of the ascarosides into the polymeric structure through the formation of covalent bonds (e.g. via condensation or transesterification) for example, to one or more carboxylic acid or hydroxyl groups present on the ascaroside.

The types of organic components with which the ascaroside can be compounded can vary widely. In some embodiments, the ascaroside is compounded with a material such as coir, peat, and/or pine bark. In some embodiments, the ascaroside is compounded with a polymeric resin; in such embodiments, generally any melt-processable polymer can be used to provide the organic compounded ascaroside. Certain such melt-processable polymers are biocompatible and/or biodegradable. Suitable polymers can include, but are not limited to, the types of polymers referenced herein above, as well as, e.g., polypropylene, polyethylene, poly(vinyl chloride), polystyrene, polyethylene terephthalate, polyethylene-vinyl acetate, polyurethane, acrylonitrile butadiene styrene (ABS), nylon, plant starch, cellulose, methylcellulose, cellulose acetate, polylactic acid (PLA), polyhydroxyalkanoate (PHA), and derivatives and co-polymers of any two or more thereof.

In some embodiments, an inorganic compounded ascaroside is provided. Inorganic compounding in this context may involve mixing and/or blending of an inorganic component and one or more additives (here, the one or more ascarosides and, optionally, one or more additional components, such as the types of agronomically acceptable excipients and additives referenced herein below). In some embodiments, such embodiments can be described as formulations wherein the ascaroside is impregnated, absorbed or adsorbed within the inorganic component. Mixing can be done in various manners, and can be done at room temperature or at elevated temperature and may or may not include solvents or other adjuvants. The inorganic material can vary and, in some embodiments, can comprise one or more of perlite, vermiculite, sand, clay, talc, wood flour, peat, silica gel, mica, activated charcoal, or the like. In some embodiments, the inorganic material can be a fertilizer, e.g., ammonium phosphate, ammonium sulfate, urca, muriate of potash, superphosphate, herbicides such as sodium chlorate and sodium sulfate, insoluble silica-type materials such as sand, broken walnut hulls, argillaceous materials such as clay of the montmorillonite, kaolinite, or other types, diatomaceous earths, granulated corn cobs, soapstone, quartz, and agricultural minerals such as sulfur, gypsum, lime, calcium carbonate, and the like, as well as any combination of two or more thereof.

In certain embodiments, the invention provides formulated growing media containing any of the controlled-release ascaroside compositions described herein. Examples include formulated soilless mixes such as those widely used for plant propagation and indoor horticulture (e.g. mixtures containing a defined blend of materials such as peat moss, coir fiber, vermiculite, perlite, and the like) as well as single-component media such as perlite, vermiculite, etc. Providing such growing media pre-fortified with compositions that release ascarosides over time provides growers with a simple and economical way to treat plants with ascarosides, for example to protect such plants from pathogens. The ascaroside compositions may be present in such growing media as granules specific to the purpose of releasing ascarosides, or may be incorporated as part of one of the typical components in the media (for example the ascaroside composition may be compounded with the perlite or vermiculite in such a blend).

In some embodiments, more than one approach as provided herein can be combined. For example, a structurally modified ascaroside as described above can be further physically modified by compounding the structurally modified ascaroside with an organic or inorganic material.

### Methods of Use and Compositions

The modified ascarosides described herein can find use in treating living plants, seeds, soil surrounding plants, or soil in which seeds/seedlings are to be planted. In some embodiments, the modified ascaroside is applied to a portion of a plant, e.g., one or more of a root, stem, leaf, seed, and/or flower. Such methods can be conducted at any one or more stages in the life cycle of a plant, e.g., from seed to seedling to growing plant to just prior to or after harvest.

While not the primary focus of this invention, the inventors also recognize that compositions and methods herein may have application for the treatment of animals with ascarosides (including treatment of humans with ascarosides as therapeutic agents or nutraceuticals). In particular, in certain embodiments, the present invention includes modified release formulations of ascr#7 (or derivatives or analogs thereof) for use to treat or ameliorate health disorders in animals and humans.

Advantageously, methods employing the disclosed modified ascarosides allow for modified release of ascarosides (relative to methods employing un-modified ascarosides), e.g., allowing delayed release and/or controlled release and/or extended release. By “delayed release,” it is meant that the ascaroside is not immediately provided to the plant (or animal) upon application of a compound or composition to the plant or animal or its environment (e.g., soil); rather, the ascaroside is released at a time after application (or upon certain environmental trigger(s), as referenced herein). By “controlled release” it is meant that the ascaroside is released over time, e.g., at a largely pre-determined rate. By “extended release” is meant that the ascaroside is released over a longer period of time after application to the plant, animal, or soil than would be achieved by application of a corresponding non-modified ascaroside. In some embodiments, the disclosed modified ascarosides release the one or more ascarosides substantially only upon response to an environmental trigger, e.g., conditions associated with light, temperature, soil pH, humidity, enzyme changes, and the like. Modified release as provided by modified ascarosides provided herein can, in some embodiments, allow less ascaroside to be used over the same period of activity than direct application of an un-modified ascaroside composition. In some embodiments, such controlled release can provide for decreased leaching, decreased evaporation, and decreased degradation (photolytic, hydrolytic, and/or microbial) of the ascaroside, any of which can remove or degrade the ascaroside before it can perform its desired function.

The disclosed treatment methods can, in some embodiments, protect growing plants in the manner described in U.S. Pat. No. 10,136,595, which is incorporated by reference herein in its entirety. For example, such methods can enhance pathogen resistance and/or induce one or more plant defense responses (thereby inhibiting pathogen growth and/or infestation) in a plant to (or near) which the modified ascaroside is applied. Pathogens against which the disclosed methods can enhance resistance include, but are not limited to, oomycetes, bacteria, nematodes, viruses, and insects, e.g., including but not limited to, Pseudomonas syringae, Phytophthora infestans, Blumeria graminis, Heterodera schachtii, Meloidogyne incognita, Meloidogyne hapla, and turnip crinkle virus. In some embodiments, such modified release profiles are such that the modified ascaroside may be effective for a longer period of time than a corresponding treatment with a (non-modified) ascaroside.

The disclosed treatment methods can, in some embodiments, protect plants or plant-based products from contamination with pathogens or prevent or delay the onset of spoilage of plants or produce in the manner described in patent applications PCT/US22/81895 and PCT/US23/17947 each of which is incorporated by reference herein in its entirety.

The exact method by which a plant, seed or soil is treated with a modified ascaroside is not particularly limited. Treatment of seeds, plants and/or soil according to the present disclosure can be carried out, e.g., by immersion, spraying, evaporation, fogging, scattering, painting on, side dressing, or in-furrow application. For example, in certain embodiments, plants or soil can be sprayed with a suitable liquid composition, a solid plastic mulch composition containing ascarosides as described above can be applied on soil around plants, plants can be grown or propagated in a medium containing any of the modified compositions describe herein (or in a pot or container formed from a composition as described herein) and/or a granular composition can be provided for in-furrow application or side-dressing.

The types of plants that can be treated according to the presently disclosed methods is not particularly limited and can be, for example, fruit and vegetable plants, trees, and shrubs. Non-limiting examples of plants that can be treated according to the disclosed methods include, but are not limited to, plants selected from the group consisting of tobacco, Arabidopsis, tomato, barley, potato, sweet potato, yam, cotton, soybean, strawberry, sugar beet, com, rice, wheat, rye, oat, sorghum, millet, bean, pea, apple, banana, pear, cherry, peach, plum, apricot, almond, grape, kiwi, mango, melon, papaya, walnut, hazelnut, pistachio, raspberry, blackberry, loganberry, blueberry, cranberry, orange, lemon, grapefruit, tangerine, lettuce, carrots, onions, broccoli, cabbage, avocado, cocoa, cassava, cotton, and flax.

In the methods provided herein, modified ascarosides can be directly applied to the plant and/or soil or can be formulated into a composition that can be applied to the plant and/or soil. As such, the present disclosure provides compositions that generally comprise at least one modified ascaroside (including, e.g., structurally modified ascarosides and/or physically modified ascarosides) and one or more inert ingredients, e.g., one or more agronomically acceptable carriers. It is preferred that non-toxic carriers be used in the methods of the present invention.

The term “agronomically acceptable carrier” includes any carrier suitable for administration to a plant or soil, e.g., customary excipients in formulation techniques, such as used to form solutions (e.g., directly sprayable or dilutable solutions), emulsions, (e.g., emulsion concentrates and diluted emulsions), wettable powders, suspensions, soluble powders, powders, dusts, pastes, soluble powders, granules, suspension-emulsion concentrates, encapsulation into polymeric materials, coatable pastes, natural and synthetic materials impregnated with active compound and microencapsulations in polymeric substances. These compositions can be produced in a known manner, for example, by mixing the modified ascaroside(s) with one or more agronomically acceptable carriers, such as liquid solvents or solid carriers, optionally with the use of additional components including, but not limited to, surfactants, including emulsifiers, dispersants, foam-formers, colorants, processing aids, lubricants, fillers, reinforcements, flame retardants, light stabilizers, ultraviolet radiation absorbers, weather stabilizers, plasticizers, release agents, perfumes, heat-retaining additives (e.g., silica), cross-linking agents, antioxidants, anti-foaming agents, buffers, pH modifiers, compatibility agents, drift control additives, extenders/stickers, tackifiers, plant penetrants, safeners, spreaders, wetting agents, and the like.

In some embodiments, the at least one modified ascaroside is the only active agent within the composition. In some embodiments, the composition includes one or more additional ascarosides (e.g., in another modified form or in unmodified form). In some embodiments, one or more other active agents are included within the composition (e.g., one or more pesticides, fertilizers, etc.). The modified ascaroside-containing compositions provided herein can be in various forms, including solid and liquid forms.

If the agronomically acceptable carrier is water, in some embodiments, an organic solvent may be incorporated as an auxiliary liquid solvent. Suitable liquid solvents include, for example, aromatics (e.g., xylene, toluene and alkylnaphthalenes); chlorinated aromatics or chlorinated aliphatic hydrocarbons (e.g., chlorobenzenes, chloroethylenes and methylene chloride); aliphatic hydrocarbons (e.g., cyclohexane); paraffins (e.g., petroleum fractions, mineral and vegetable oils); alcohols (e.g., butanol or glycol and their ethers and esters); ketones (e.g., acetone, methyl ethyl ketone, methyl isobutyl ketone and cyclohexanone) and strongly polar solvents (e.g., dimethylformamide and dimethyl sulfoxide).

Suitable solid agronomically acceptable carriers include, for example, ammonium salts and ground natural minerals (e.g., kaolins, clays, talc, chalk, quartz, attapulgite, montmorillonite and diatomaccous earth); ground synthetic minerals (e.g., highly disperse silica, alumina and silicates); crushed and fractionated natural rocks (e.g., calcite, marble, pumice, sepiolite and dolomite); synthetic granules of inorganic and organic meals; granules of organic material (e.g., sawdust, coconut shells, maize cobs and tobacco stalks).

Suitable emulsifiers and foam-formers include, for example, nonionic and anionic emulsifiers (e.g., polyoxyethylene fatty acid esters, polyoxyethylene fatty alcohol ethers, for example, alkylaryl polyglycol ethers, alkylsulfonates, alkyl sulphates and arylsulfonates) protein hydrolysates.

Suitable dispersants include, for example, lignin-sulfite waste liquors and methylcellulose. Tackifiers such as carboxymethylcellulose and natural and synthetic polymers in the form of powders, granules or lattices, such as gum arabic, polyvinyl alcohol and polyvinyl acetate, as well as natural phospholipids, such as cephalins and lecithins, and synthetic phospholipids, can be used in the disclosed compositions. Other additives may include, for example, mineral and vegetable oils.

Colorants such as inorganic pigments, for example, iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc may also be included in the compositions.

Methods of preparing solid and liquid compositions for agrichemical use are generally known and can be employed according to the present disclosure (where such methods involve incorporating one or more modified ascarosides) within such compositions). As referenced above, compositions according to the present disclosure can, in some embodiments, be in the form of granular material (including dusts, pellets, soluble powders, flowable powders, water-dispersible granules, and the like). In some embodiments, compositions according to the present disclosure can be in liquid form (e.g., solutions, suspensions, or emulsions). Some modified ascarosides may have limited solubility in water; as such, in some embodiments, the ascaroside or modified ascaroside is dissolved in a water-miscible co-solvent (e.g., ethanol) to initially dissolve the modified ascaroside. Subsequently, in some embodiments, the dissolved modified ascaroside can be added to a suitable amount of water to provide an aqueous modified ascaroside-containing composition. In some embodiments, compositions are in the form of a granular material treated with a modified ascaroside-containing liquid. In some embodiments, a composition comprising a modified ascaroside is formed into fibers or filaments and in some such embodiments, a woven or non-woven textile (e.g., film) can be produced therefrom. In some embodiments, a composition as provided herein is pelletized. In some embodiments, a composition as provided herein is in the form of a film, e.g., plastic mulch. In some embodiments, a composition as provided herein is in the form of a molded article, for example a pot, container, bag or sleeve in which plants can be propagated or grown. Any of the solid compositions provided herein can optionally be coated via methods generally known in the art to further delay release of the modified ascaroside.

In some embodiments, methods are provided of producing compositions wherein an ascaroside is compounded with a polymer (e.g. to form a film, fiber, pellet, molded or extruded article, or filament). Such compounding generally comprises heating or melting the polymer and blending the softened or molten polymer with the ascaroside. Such methods can produce a physical mixture of the ascaroside and the polymer, or can produce a copolymer (e.g. by processes such as reactive extrusion) wherein an ascaroside reacts with the polymer (e.g. via transesterification or condensation) with the polymer during the compounding process.

It is contemplated that compounds, compositions, and methods of the present application encompass variations and adaptations developed using information from the embodiments described in the present disclosure. Adaptation or modification of the methods and processes described in this specification may be performed by those of ordinary skill in the relevant art.

It will be appreciated that use of headers in the present disclosure are provided for the convenience of the reader. The presence and/or placement of a header is not intended to limit the scope of the subject matter described herein. Unless otherwise specified, embodiments located in one section of the application apply throughout the application to other embodiments, both singly and in combination.

Throughout the description, where compositions, compounds, or products are described as having, including, or comprising specific components, or where processes and methods are described as having, including, or comprising specific steps, it is contemplated that, additionally, there are articles, devices, and systems of the present application that consist essentially of, or consist of, the recited components, and that there are processes and methods according to the present application that consist essentially of, or consist of, the recited processing steps.

It should be understood that the order of steps or order for performing certain action is immaterial so long as the described method remains operable. Moreover, two or more steps or actions may be conducted simultaneously.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A structurally modified ascaroside, selected from the group consisting of:
a) an ascaroside-based oligomer or polymer, comprising two or more ascaroside moieties, optionally comprising linker moieties between adjacent ascaroside moieties;
b) an oligomer- or polymer-functionalized ascaroside, comprising an ascaroside moiety with one, two, or three oligomers or polymers attached thereto; and
c) an ascaroside-pendant polymer, comprising a polymer with one or more pendant ascaroside moieties attached thereto, optionally comprising linker moieties between the polymer and the pendant ascaroside moieties.

2. The structurally modified ascaroside of claim 1, wherein the modified ascaroside is an ascaroside-based oligomer or polymer and wherein the oligomer or polymer comprises two or more ascaroside moieties with no linker moieties between adjacent ascaroside moieties.

3. The structurally modified ascaroside of claim 2, wherein adjacent ascaroside moieties are attached to one another via an ester linkage formed from a hydroxyl group on a first ascaroside molecule and a carboxylic acid on a second ascaroside molecule.

4. The structurally modified ascaroside of claim 1, wherein the modified ascaroside is an ascaroside-based oligomer or polymer, and wherein the oligomer or polymer comprises two or more ascaroside moieties with linker moieties between adjacent ascaroside moieties.

5. The structurally modified ascaroside of claim 4, wherein the linker moieties are selected from the group consisting of ether, ester, amide, glycosidic, urethane, carbonate, and siloxane moieties.

6. The structurally modified ascaroside of any of claims 1-5, wherein the modified ascaroside is an ascaroside-based oligomer or polymer wherein the oligomer or polymer comprises two to one thousand ascaroside moieties.

7. The structurally modified ascaroside of claim 6, wherein the oligomer or polymer comprises two to five hundred ascaroside moieties.

8. The structurally modified ascaroside of claim 6, wherein the oligomer or polymer comprises two to one hundred ascaroside moieties.

9. The structurally modified ascaroside of claim 6, wherein the oligomer or polymer comprises two to ten ascaroside moieties.

10. The structurally modified ascaroside of claim 6, wherein the oligomer or polymer comprises at least ten ascaroside moieties.

11. The structurally modified ascaroside of claim 6, wherein the oligomer or polymer comprises at least twenty ascaroside moieties.

12. The structurally modified ascaroside of claim 6, wherein the oligomer or polymer comprises at least fifty ascaroside moieties.

13. The structurally modified ascaroside of claim 1, wherein the modified ascaroside is an oligomer- or polymer-functionalized ascaroside, comprising one oligomer or polymer attached to the ascaroside.

14. The structurally modified ascaroside of claim 1, wherein the modified ascaroside is an oligomer- or polymer-functionalized ascaroside, comprising two oligomers or polymers attached to the ascaroside.

15. The structurally modified ascaroside of claim 1, wherein the modified ascaroside is an oligomer- or polymer-functionalized ascaroside, comprising three oligomers or polymers attached to the ascaroside.

16. The structurally modified ascaroside of any of claims 13-15, wherein the oligomer or polymer comprises a biodegradable polymer.

17. The structurally modified ascaroside of any of claims 13-15, wherein the oligomer or polymer is selected from the group consisting of chitosan, chitin, starches, lignin, agar, pectin, cellulose, other polysaccharides (e.g., bark, sawdust, other cellulosic waste), poly(ethylene glycol), poly(caprolactone), poly(lactide), poly(glycolic acid), polybutylene, poly(vinyl alcohol), poly(vinyl chloride), poly(citric acid), poly(acrylate), poly(aspartic acid), poly(ethylene), polyethylene vinyl acetate (PEVA), polystyrene, divinyl benzene, and derivatives and copolymers thereof.

18. The structurally modified ascaroside of claim 1, wherein the modified ascaroside is an ascaroside-pendant polymer, comprising about five to about five hundred pendant ascaroside moieties attached thereto.

19. The structurally modified ascaroside of claim 1, wherein the modified ascaroside is an ascaroside-pendant polymer, and wherein the one or more pendant ascaroside moieties are directly bonded to the polymer.

20. The structurally modified ascaroside of claim 1, wherein the modified ascaroside is an ascaroside-pendant polymer, comprising linker moieties between the polymer and the one or more pendant ascaroside moieties.

21. The structurally modified ascaroside of claim 20, wherein the linker moieties are selected from the group consisting of ether, ester, amide, glycosidic, urethane, carbonate, and siloxane moieties.

22. The structurally modified ascaroside of any one of claims 1-21, wherein the ascaroside moieties have the structure (I) 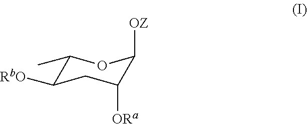
where:
Z is an optionally substituted C₃₋₄₀ aliphatic group, and
each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g.—a moiety —C(O)OR^{c}), a C₂₋₂₀ carbamate (e.g.—a moiety) —C(O)N(R^{c})₂), a C₂₋₂₀ thioester (e.g. a moiety —C(S)R^{c}), a C₂₋₂₀ thiocarbonate (e.g. a moiety —C(S)OR^{c}), a C₂₋₂₀ dithiocarbonate (e.g. a moiety —C(S)SR^{c}), a C₁₋₂₀ thiocarbamate (e.g. a moiety —C(S)NR^{c})₂), a sugar moiety, a peptide, a polymer chain, or a linkage via a bond or a carbon-containing linker moiety to another ascaroside molecule. Where R^{c} is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule, and where R^{a} and R^{b} may be taken together to form an optionally substituted ring, optionally containing one or more heteroatoms, and optionally containing one or more sites of unsaturation.

23. The structurally modified ascaroside of claim 22, wherein Z is selected from the group consisting of:
(i) —CH(CH₃)—R¹, where R¹ is an optionally substituted C₁₋₄₀ aliphatic group;
(ii) —CH(CH₃)—(CH₂)ₙ—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(iii) —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(iv) —CH(CH₃)—(CH₂)ₙ—CH(OH)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(v) —CH(CH₃)—(CH₂)ₙ—C(O)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(vi) —(CH₂)ₙ—CO₂R², where n is an integer from 1 to 40, and R² is is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(vii) —(CH₂)ₙ—CH═CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
(viii) —(CH₂)ₙ CH(OH)CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule; and
(ix) —(CH₂)ₙ—C(O)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule.

24. The structurally modified ascaroside of claim 22, wherein Z is selected from the group consisting of:
(x) —CH(CH₃)—(CH₂)ₙ—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xi) —CH(CH₃)—(CH₂)ₙ—CH═CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xii) —CH(CH₃)—(CH₂)ₙ—CH(OH)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xiii) —CH(CH₃)—(CH₂)ₙ—C(O)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xiv) —(CH₂)ₙCON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xv) —(CH₂)ₙ—CH—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
(xvi) —(CH₂)ₙ—CH(OH)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule; or
(xvii) —(CH₂)ₙ—C(O)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule.

25. The structurally modified ascaroside of any of claims 22 to 24, wherein R^{a} and R^{b} are each —H.

26. The structurally modified ascaroside of any one of claims 22 to 25, wherein Z is —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

27. The structurally modified ascaroside of any of claims 1-26, wherein at least one of the ascaroside moieties is ascr#18.

28. A physically modified ascaroside, comprising one or more ascarosides compounded with an organic or inorganic material.

29. The physically modified ascaroside of claim 28, wherein the ascaroside is compounded with an organic material selected from coir, peat, and/or pine bark.

30. The physically modified ascaroside of claim 28, wherein the ascaroside is compounded with a polymeric resin.

31. The physically modified ascaroside of claim 30, wherein the polymeric resin is a biodegradable polymer.

32. The physically modified ascaroside of claim 30, wherein the polymeric resin is a melt-processable polymer.

33. The physically modified ascaroside of claim 30, wherein the polymeric resin is selected from the group consisting of polypropylene, polyethylene, poly(vinyl chloride), polystyrene, polyethylene terephthalate, polyethylene-vinyl acetate, polyurethane, acrylonitrile butadiene styrene (ABS), nylon, plant starch, polylactic acid (PLA), polyhydroxyalkanoate (PHA), and derivatives and co-polymers of any two or more thereof.

34. The physically modified ascaroside of claim 28, wherein the ascaroside is compounded with an inorganic material selected from the group consisting of perlite, vermiculite, sand, clay, talc, wood flour, peat, silica gel, mica, activated charcoal, and combinations thereof.

35. The physically modified ascaroside of claim 28, wherein the ascaroside is compounded with an inorganic material selected from a fertilizer (e.g., ammonium phosphate, ammonium sulfate, urea, muriate of potash, superphosphate), herbicides (e.g., sodium chlorate or sodium sulfate), insoluble silica-type material (e.g., sand or broken walnut hulls), argillaceous materials (e.g., clay of the montmorillonite, kaolinite, or other type), diatomaceous earths, granulated corn cobs, soapstone, quartz, lead, iron, and agricultural minerals (e.g., sulfur, gypsum, lime, calcium carbonate, and the like), and combinations thereof.

36. The physically modified ascaroside of any of claims 28-35, comprising one or more bonds between one or more of the ascarosides and the organic or inorganic material.

37. The physically modified ascaroside of any of claims 28-35, further comprising one or more additional components selected from the group consisting of surfactants, including emulsifiers, dispersants, foam-formers, colorants, processing aids, lubricants, fillers, reinforcements, flame retardants, light stabilizers, ultraviolet radiation absorbers, weather stabilizers, plasticizers, release agents, perfumes, heat-retaining additives (e.g., silica), cross-linking agents, antioxidants, anti-foaming agents, buffers, pH modifiers, compatibility agents, drift control additives, extenders/stickers, tackifiers, plant penetrants, safeners, spreaders, and wetting agents.

38. The physically modified ascaroside of any one of claims 28-37, wherein at least one of the ascarosides has the structure (I) 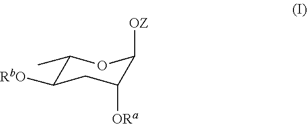
where:
Z is an optionally substituted C₃₋₄₀ aliphatic group, and each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g. —a moiety —C(O)OR^{c}), a C₂₋₂₀ carbamate (e.g. —a moiety —C(O)N(R^{c})₂), a C₂₋₂₀ thioester (e.g. a moiety) —C(S)R^{c}, a C₂₋₂₀ thiocarbonate (e.g. a moiety —C(S)OR^{c}), a C₂₋₂₀ dithiocarbonate (e.g. a moiety —C(S)SR^{c}), a C₁₋₂₀ thiocarbamate (e.g. a moiety) —C(S)N(R^{c})₂), a sugar moiety, a peptide, a polymer chain, or a linkage via a bond or a carbon-containing linker moiety to another ascaroside molecule. Where R^{c} is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule, and where R^{a} and R^{b} may be taken together to form an optionally substituted ring, optionally containing one or more heteroatoms, and optionally containing one or more sites of unsaturation.

39. The physically modified ascaroside of claim 38, wherein Z is selected from the group consisting of:
i. —CH(CH₃)—R¹, where R¹ is an optionally substituted C₁₋₄₀ aliphatic group;
ii. —CH(CH₃)—(CH₂)ₙ—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
iii. —CH(CH₃)—(CH₂)ₙ—CH═CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
iv. —CH(CH₃)—(CH₂)ₙ—CH(OH)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
v. —CH(CH₃)—(CH₂)ₙ—C(O)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
vi. —(CH₂)ₙ—CO₂R², where n is an integer from 1 to 40, and R² is is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
vii. —(CH₂)ₙ—CH═CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule;
viii —(CH₂)ₙCH(OH)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule; and
ix. —(CH₂)ₙ—C(O)—CH—CO₂R², where n is an integer from 1 to 40, and R² is —H, a metal cation, an optionally substituted C₁₋₂₀ aliphatic group, an optionally substituted C₁₋₂₀ heteroaliphatic group, an optionally substituted aromatic group, an optionally substituted heteroaryl group, a glycoside, an amino acid, a peptide, a nucleotide, or a linkage via a bond or a carbon-containing linker moiety to another ascaroside molecule.

40. The physically modified ascaroside of claim 38, wherein Z is selected from the group consisting of:
x. —CH(CH₃)—(CH₂)ₙ—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xi. —CH(CH₃)—(CH₂)ₙ—CH═CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xii. —CH(CH₃)—(CH₂)ₙ—CH(OH)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xiii. —CH(CH₃)—(CH₂)ₙ—C(O)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xiv. —(CH₂)ₙ—CON(R³)₂, where n is an integer from 1 to 40, and each R³ is independently —H, an optionally substituted C₁₋₂₀ aliphatic group, an optionally substituted C¹⁻²⁰ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xv. —(CH₂)ₙ—CH═CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule;
xvi. —(CH₂)ₙ—CH(OH)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule; or
xvii. —(CH₂)ₙ—C(O)—CH—CON(R³)₂, where n is an integer from 1 to 40, and each R³ is independently —H, an optionally substituted C₁₋₂₀ aliphatic group, an optionally substituted C₁₋₂₀ heteroaliphatic group, an optionally substituted aromatic group, an optionally substituted heteroaryl group, a polymer chain, an amino acid, a peptide, a nucleotide, or a linkage via a bond or a carbon-containing linker moiety to another ascaroside molecule.

41. The physically modified ascaroside of claim 38, wherein R^{a} and R^{b} are each —H.

42. The physically modified ascaroside of any one of claims 38-41, wherein Z is —CH(CH₃)—(CH₂)ₙ—CO₂R², where n is an integer from 1 to 40, and R² is —H, a metal cation, an optionally substituted C₁₋₂₀ aliphatic group, an optionally substituted aromatic group, a glycoside, an amino acid, a peptide, or a nucleotide.

43. The physically modified ascaroside of any of claims 28-42, wherein at least one of the ascaroside moieties is ascr#18.

44. A composition comprising the structurally modified ascaroside of any of claims 1-27 and/or the physically modified ascaroside of any of claims 28-42 and one or more agronomically acceptable excipients or additives.

45. The composition of claim 44, in the form of a liquid.

46. The composition of claim 45, wherein the liquid is a sprayable formulation.

47. The composition of claim 44, in the form of a solid.

48. The composition of claim 47, wherein the solid comprises granules.

49. The composition of claim 47, wherein the solid comprises plastic mulch.

50. The composition of claim 47, wherein the solid composition is a film.

51. A method, comprising:
contacting a plant or portion thereof or soil surrounding the plant or portion thereof with the structurally modified ascaroside of any of claims 1-27, the physically modified ascaroside of any of claims 28-43, or the composition of any of claims 42-48,
wherein the ascaroside or composition is effective to increase plant resistance to one or more pathogens and/or to induce an immune response in the plant or portion thereof.

52. The method of claim 51, wherein the portion thereof is selected from the group consisting of root, stem, leaf, seed, and flower.

53. The method of claim 51 or 52, wherein the plant is a vegetable or fruit plant.
